SEQUENCE LISTING (14 sequences)

SEQ ID NO: 1 - DNA, 19 bp, Artificial Sequence
  STAT 3 siRNA 1
agtcgaatgttctctatca

SEQ ID NO: 2 - DNA, 19 bp, Artificial Sequence
  STAT 3 siRNA2
ggcgtccagttcactacta

SEQ ID NO: 3 - DNA, 19 bp, Artificial Sequence
  STAT 3 siRNA 4
gcgtccatcctgtggtaca

SEQ ID NO: 4 - DNA, 60 bp, Artificial Sequence
  Oligonucleotide
gatccccagtcgaatgttctctatcattcaagagatgatagagaacattcgacttttttc

SEQ ID NO: 5 - DNA, 60 bp, Artificial Sequence
  Oligonucleotide
tcgagaaaaaagtcgaatgttctctatcatctcttgaatgatagagaacattcgactggg

SEQ ID NO: 6 - DNA, 60 bp, Artificial Sequence
  Oligonucleotide
gatccccggcgtccagttcactactattcaagagatagtagtgaactggacgcctttttc

SEQ ID NO: 7 - DNA, 60 bp, Artificial Sequence
  Oligonucleotide
tcgagaaaaaggcgtccagttcactactatctcttgaatagtagtgaactggacgccggg

SEQ ID NO: 8 - DNA, 60 bp, Artificial Sequence
  Oligonucleotide
gatccccgcgtccatcctgtggtacattcaagagatgtaccacaggatggacgctttttc

SEQ ID NO: 9 - DNA, 60 bp, Artificial Sequence
  Oligonucleotide
tcgagaaaaagcgtccatcctgtggtacatctcttgaatgtaccacaggatggacgcggg

SEQ ID NO: 10 - DNA, 15 bp, Artificial Sequence
  decoy oligo
catttcccgtaaatc

SEQ ID NO: 11 - DNA, 15 bp, Artificial Sequence
  decoy oligo
gtaaagggcatttac

SEQ ID NO: 12 - DNA, 19 bp, Artificial Sequence
  STAT 3 siRNA 3
gattgggcatatgcggcca

SEQ ID NO: 13 - DNA, 60 bp, Artificial Sequence
  siRNA 3 1
gatccccgattgggcatatgcggccattcaagagatggccgcatatgcccaatctttttc

SEQ ID NO: 14 - DNA, 60 bp, Artificial Sequence
  si RNA 3 2
tcgagaaaaagattgggcatatgcggccatctcttgaatggccgcatatgcccaatcggg

(19) United States
(12) Patent Application Publication
(10) Pub. No.: US 2010/0035793 A1
(43) Pub. Date: Feb. 11, 2010

(54) Modulators

(76) Inventors: Cheh Peng Lim, Singapore (SG); Xinmin Cao, Singapore (SG); Ivor Jiun Lim, Singapore (SG); Toan-Thang Phan, Singapore (SG)

Correspondence Address: JACOBSON HOLMAN PLLC, 400 SEVENTH STREET N.W., SUITE 600, WASHINGTON, DC, 20004, US

(21) Appl. No.: 11/989,244

(22) PCT Filed: Jul. 27, 2005
(86) PCT No.: PCT/SG2005/000252

§ 371 Date: Jan. 23, 2008

(51) Int. Cl. A61K 38/02; C07H 21/00; C07J 1/00; C07K 2/00; A61K 31/7052; A61K 31/56; C12Q 1/02; A61P 17/02

(52) U.S. Cl. 514/2; 536/24.5; 552/502; 536/23.1; 530/300; 514/44 A; 514/178; 435/29; 514/44 R

(57) ABSTRACT

Use of a composition that modulates a STAT in the manufacture of a medicament for treating or preventing a fibroproliferative disease. The fibroproliferative disease may comprise keloid scarring. The composition may modulate one or more of; activity; phosphorylation; level of expression; or sub-cellular localisation of the STAT. The STAT may be STAT 3.

The present invention relates to compositions and pharmaceutical compositions for treating fibroproliferative disease.

Excessive fibroblast proliferation and the deposition of excess collagen are characteristics of fibroproliferative diseases such as scleroderma, pulmonary fibrosis, keloid scaring and surgical adhesions among others. One potential treatment of fibroproliferative disease is inhibitors of sulfated glycosaminoglycans (GAG). It is known that inhibitors of GAG demonstrated inhibition of fibroproliferative tissue and decreased collagen expression in several fibroproliferative disease models. However, currently there appear to be no suitable treatments in use for fibroproliferative diseases.

Wound healing is a regulated yet a complex event including the breakdown of fibrin clots, degradation of extracellular matrix (ECM), promotion of angiogenesis, and the migration and proliferation of cells. In the case of cutaneous injury keratinocytes and fibroblasts are the cells found to proliferate and migrate cocktails of chemokines, cytokines, and growth factors, are secreted temporally and spatially to direct appropriate response from neutrophils, macrophages, keratinocytes, and fibroblasts to facilitate normal wound healing the event of excessive cutaneous wound healing, scar formation occurs, ranging from hypertrophic scars to keloid scars. Keloid scars represent the most extreme example of cutaneous scarring that uniquely afflicts humans, a pathological response to wound healing with largely effete treatment strategies due to its recurrent nature. The term “keloid” was coined in 1806 to describe the crab claw-like appearance of the scar. Keloid scars are defined as scars that spread beyond the boundaries of the original wound and do not regress spontaneously. In contrast, hypertrophic scars do not develop beyond the periphery of wound, with spontaneous softening and flattening. Keloid scars afflict humans exclusively, and inciting skin trauma can range from small injuries such as ear piercing and abrasions, to more severe trauma such as surgery or burns.

Keloid scars are aggressive in nature and seem to be more common in Africans and Asians. Treatment and management of keloid scars have been difficult due to their recurrence, and although a myriad of treatments are available, none alone is satisfactory. Known treatments for a keloid scar gives the best results if started soon after the keloid appears. Available treatments include:
Removal with conventional surgery is an unreliable technique. It requires great care, as keloids that return after being removed may be larger than the original. Keloids return in more than 45 percent of people when they are removed surgically (Cosman et al. 1961);
Moist wound coverings or dressings made of silicone gel sheets have been shown in studies to reduce keloid prominence over time. This treatment is safe and painless;
Injections with Corticosteroid such as triamcinolone acetonide or another corticosteroid medicine typically are repeated at intervals of four to six weeks. This treatment may reduce keloid size and irritation, but injections are uncomfortable;
Compression using a bandage or tape to apply continuous pressure 24 hours a day for a period of six to 12 months. Such compression can provide a thinning effect on the skin;
Cryosurgery or freezing treatment with liquid nitrogen is repeated every 20 to 30 days. It can cause a side effect of lightening the skin color, which limits this treatment's usefulness (Alster & Tanzi 2003);
Radiation therapy is controversial because radiation increases the risk of cancer. Radiation treatments may reduce scar formation if they are used soon after a surgery, during the time a surgical wound is healing (Ragoowansi et al. 2001);
Laser therapy is an alternative to conventional surgery for keloid removal. There is no good evidence that keloids are less likely to recur after laser therapy than after regular surgery (Alster & Tanzi 2003);
Experimental treatments with medicines such as various types of interferon and 5-fluorouracil and bleomycin have also been tested.

These treatments are largely unreliable and require great care. Keloid scars return in many cases and may be larger than the original scar.

Keloid scars are characterized by excessive deposition of collagen and other ECM components such as fibronectin by dermal fibroblasts, and increased fibroblast proliferation upon wounding. There are conflicting reports on the mRNA expression levels of procollagen in keloid scars. Overproduction of type I procollagen mRNA without alteration of type III procollagen mRNA level was shown to reverse the ratio of type I/III procollagen mRNA in keloid fibroblast compared to normal fibroblast cultures (Uitto et al. 1985 and Abergel et al. 1985). However, a later publication reported that procollagen III mRNA was also upregulated to 20-fold in keloid tissues (Naitoh et al. 2001). Elevated IL-6 (Xue et al. 2000) and VEGF (Wu et al. 2004) expressions have also been observed in keloid fibroblasts.

The exact mechanism for keloid pathogenesis due to cutaneous injury is still unclear. Increased levels of cytokines and growth factors, or their receptors, and sensitization of mitogenic response to these secreted factors after wounding in keloid fibroblasts have been suggested. TGF-β1 and TGF-β2, but not TGF-β3, were elevated in keloid fibroblasts (Lee et al. 1999), and increased sensitivity to TGF-β1 in keloid fibroblasts increased fibronectin (Babu et al. 1992) and collagen production, as well as cell proliferation. PDGF a receptor expression was enhanced in keloid fibroblasts, which corresponded to increased mitotic response in response to all three PDGF isoforms (Haisa et al. 1994). Overexpresison of IGF-1 receptor detected in keloid fibroblasts enhanced their invasiveness, but not fibroproliferation (Yoshimoto et al. 1999). Besides dermal fibroblasts, overlying epidermis of keloid tissues also showed excessive cytokine expression, such as VEGF (Gira et al 2004). In addition, aberrant epidermal-mesenchymal interactions have also been implicated in keloid pathogenesis, evidenced by increased proliferation (Lim et al. 2001) and secretion of collagen I and III (Lim et al. 2002) in normal fibroblasts when cocultured with keloid-derived keratinocytes compared to coculture with normal human keratinocytes. Keloid fibroblasts cocultured with keloid keratinoytes also showed increased expression of TGF-β1, TGF-β2, TGF-β receptor 1, Smad2, type I collagen, connective tissue growth factor (CTGF), and IGF-II receptor, while keloid keratinocytes cocultured with keloid fibroblasts showed increased TGF-β1, TGF-β3, and TGF-β receptor 1 expression (Xia et al. 2004). Mutations in regulatory genes have also been proposed in scar fibrosis, such as p53 (Saed et al. 1998).

STAT proteins perform the dual function of Signal Transduction and Activation of Transcription. STAT proteins are a family of cytoplasmic proteins that function as secondary messengers and transcription factors. They play a fundamental role in normal cell signaling in response to cytokines and growth factors. There are seven STAT proteins currently known in mammals (STAT 1, 2, 3, 4, 5a, 5b, and 6) each encoded in a distinct gene but subject to alternative RNA splicing or post-translational proteolytic processing which account for additional variants of STAT in malignant cells. Most notably STAT 3 and 5 are found constitutively activated in breast cancer and head & neck cancer, melanoma, multiple myeloma, among other malignancies. Aberrant signaling through a number of upstream pathways can result in constitutively activated STAT in tumor cells, leading to malignant progression via a few common biologic mechanisms involving prevention of apoptosis by upregulation of the Bcl gene expression and dysregulation of cell-cycle progression by upregulation of Cyclin D1 gene expression. It is known that inhibition of STAT3 is effective at blocking cancer and has been shown to work effectively against head and neck cancer (Leong et al. 2003) and skin cancer (Chan et al. 2004 and Pedranzini et al. 2004).

Signal transducers and activators of transcription 3 (STAT 3) is a latent transcription factor that is involved in diverse processes, including cell proliferation and migration, inflammation, immune response, and cell survival. It is activated by various growth factors and cytokines (Darnell 1997) by obligatory Tyr705 phosphorylation which enables the dimerization via its phosphotyrosine residue and reciprocal SH2 domain of its dimmer partner. STAT 3 can homodimerize or heterodimerize with Stat1, and the dimer then translocates to the nucleus to bind to target DNA sequence, effecting the transcription of target genes (Darnell et al 1994). While Tyr705 phosphorylation is essential for STAT 3 activation, Ser727 phosphorylation was shown to be required for maximal gene activation (Wen et al 1995). For cytokine receptors that lack intrinsic tyrosine kinase activity, Tyr705 phosphorylation was achieved by receptor-associated Janus kinases (Jaks) (Darnell 1997). STAT 3 knockout resulted in embryonic lethality (Takeda et al. 1997), prompting further conditional tissue or cell-specific knockout analyses (Takeda et al. 2000). It was previously reported that STAT 3-disrupted keratinocytes displayed delayed wound healing. In particular, using Cre recombinase driven by keratin 5-specific promoter, STAT 3 expression disrupted in keratinocytes showed impaired wound healing and growth factor-dependent migration of STAT 3-deficient epidermal cells in mice, although development of epidermis and hair follicles appeared normal, and proliferation remained unaffected (Sano et al 1999). Recently, overexpression of Stat3C in A549 carcinoma cells followed by microarray analyses revealed that Stat3 regulated genes are common to both wound healing and cancer, including cell invasion/migration, angiogenesis, and remodeling of ECM (Dauer et al. 2005).

A number of studies have implicated STAT activation, particularly STAT 3 in tumor progression. Strategies have been used to block the action of STAT proteins in tumor cells, including antisense methods (U.S. Pat. No. 6,727,064), interfering peptides such as the low molecular weight composition STA-21 (Song et al 2005) and phosphototyrosyl peptides (Turkson et al 2001), STAT 3 decoy oligonucleotide (Leong et al 2003), and Cucurbitacin compositions such as Cucurbitacin I (Blaskovich et al 2003) and Cucurbitacin Q (Blaskovich et al 2005).

We investigated keloid scar pathogenesis by examining skin tissues, keratinocytes and fibroblasts from normal and keloid patients.

In summary, we have shown for the first time, a novel role of STAT in the pathogenesis of fibroproliferative disease.

A first aspect of the invention provides the use of a composition that modulates a STAT in the manufacture of a medicament for treating or preventing a fibroproliferative disease.

Preferably the fibroproliferative disease comprises keloid scarring.

Preferably the term modulates refers to the activation, inhibition, delay, repression or interference of one or more of; the activity of STAT; the RNA splicing or posttranslational processing to STAT; the phosphorylation of STAT; the level of expression of STAT including both mRNA expression and protein expression; or the sub-cellular localisation of STAT. More preferably the term modulates refers to reduction in STAT 3 mediated signalling. Modulation of STAT may be assessed using the methods described in the examples below or by those commonly used in the art such as the methods in the papers referred to on pages 4 and 5 above.

Preferably the STAT comprises STAT 3. STAT 3 is a well known protein in biology particularly in the field of cancer (Wen et al. 1995). Some of the attributes of STAT 3 are described on page 5 above. The Genbank Accession number for human STAT 3 is NM_{—}139276.

In one embodiment preferably the composition is an SiRNA of STAT preferably STAT 3. Small interfering Ribonucleic Acid SiRNA or RNA interference (RNAi) has emerged as a novel cellular mechanism regulating gene expression at the post-transcriptional level and as a powerful tool to control gene function. Improved criteria for selecting effective siRNA sequences, and the generation of vectors for the delivery of siRNAs for silencing of genes in mammalian cells, tissues and animals have developed. There are several methods for preparing siRNA, such as chemical synthesis, in vitro transcription, siRNA expression vectors, and PCR expression cassettes. Irrespective of which method one uses, the first step in designing a siRNA is to choose the siRNA target gene site. The selection of an optimal siRNA results from the in vitro testing of several siRNA designed to specifically target a gene. Usually, such in vitro tests consist in the transfection of the several siRNA duplexes in a cell expressing stably the gene of interest (Nencioni et al. 2004). Specifically designed SiRNA may be able to modulate levels of STAT expression.

Preferably the SiRNA of STAT 3 comprises SEQ ID 1, SEQ ID 2 or SEQ ID 3 (shown below). These were designed against STAT 3 from the sequence STAT 3 Genbank Accession number NM_{—}139276. The STAT 3 sequence is 4978 base pairs long. 3 sequences along the STAT 3 sequence which inhibit STAT 3 in an SiRNA system have been identified. The 3 targets are named: STAT 3 siRNA 1 (nt. 461-480),

SEQ ID 1: AGTCGAATGTTCTCTATCA,;
STAT 3 siRNA 2 (nt. 1264-1283),
SEQ ID 2 GGCGTCCAGTTCACTACTA;
and
STAT 3 siRNA 4 (nt. 1662-1681)
SEQ ID 3 GCGTCCATCCTGTGGTACA.

In one embodiment the SiRNA of STAT 3 comprises SEQ ID 4 and or 5, or SEQ ID 6 and or 7 or SEQ ID 8 and or 9. A hairpin loop is conserved and flanked upstream by one of the 3 target sequences SEQ ID 1, or SEQ ID 2, or SEQ ID 3 in the sense orientation (underlined) mentioned above and down stream by a complementary anti-sense sequence (italics). Excess base pairs may then be added to the 3′ and 5′ ends preferably T rich sequences. The corresponding primers create an SiRNA hairpin loop to be broken by RNAi. It would be understood by those in the field that as long as the sequences flanking the hairpin sequence are complementary to a 19 base pair section of the STAT 3 mRNA then the SiRNA is expected to interfere with the translation of STAT 3. A 9-nucleotide short sequence was used in this case to form the hairpin loop (bold), however, various research groups have reported successful gene silencing results using hairpin siRNAs with loop size ranging between 3 to 23 nucleotides. The following is a summary of loop size and specific loop sequences used by various research groups:

!Loop Size? ?!(# of Nucleotides)? Specific Loop Sequence

| | |
|---|---|
| 3 | AUG |
| 3 | CCC |
| 4 | UUCG |
| 5 | CCACC |
| 6 | CTCGAG |
| 6 | AAGCUU |
| 7 | CCACACC |
| 9 | UUCAAGAGA |
| 23 | Not reported |

SEQ ID 4: (nt. 461-480)
F15′GATCCCCAGTCGAATGTTCTCTATCATTCAAGAGATGATAGAGAAC
ATTCGACTTTTTTC3′ (Forward strand)
SEQ ID 5: (nt. 461-480)
R15′TCGAGAAAAAAGTCGAATGTTCTCTATCATCTCTTGAATGATAGA
GAACATTCGACTGGG3′, (Reverse strand)
SEQ ID 6: (nt. 1264-1283)
F2
5′GATCCCCGGCGTCCAGTTCACTACTATTCAAGAGATAGTAGTGAACTG
GACGCCTTTTTC3′ (Forward strand)
SEQ ID 7: (nt. 1264-1283)
R25′TCGAGAAAAAGGCGTCCAGTTCACTACTATCTCTTGAATAGTAGT
GAACTGGACGCCGGG3′ (Reverse strand),
SEQ ID 8: (nt. 1662-1681)
5′GATCCCCGCGTCCATCCTGTGGTACATTCAAGAGATGTACCACAGGA
TGGACGCTTTTTC3′(Forward strand),
SEQ ID 9:
5′TCGAGAAAAAGCGTCCATCCTGTGGTACATCTCTTGAATGTACCACA
GGATGGACGCGGG3′ (Reverse strand).

It would be understood by those in the art that a variant SiRNA could be used to modulate STAT 3 activity where “variant” refers to an SiRNA wherein at one or more positions there have been nucleotide insertions, deletions, or substitutions, either conservative or non-conservative, provided that such changes result in a SiRNA whose basic properties, for example modulating activity have not significantly been changed. “Significantly” in this context means that one skilled in the art would say that the properties of the variant may still be different but would not be unobvious over the ones of the original SiRNA.

In one embodiment the SiRNA of STAT 3 is selected from the group of SEQ ID 4 and or 5, SEQ ID 6 and or 7 and SEQ ID 8 and or 9. Vectors can be prepared using techniques known in the art. One example described in the examples below Vectors of the SiRNA were constructed containing STAT 3 SiRNA targets and empty vector were transfected into 293T-based Phoenix-Ampho packaging cell line for 7-9 h, and the amphotropic retroviruses were harvested 48 h later, pelleted to remove nonadherent cells and cellular debris, and filtered through a 0.45 μM cellulose acetate membrane. It would be understood by someone skilled in the art of molecular biology that many vectors and packaging cell lines are available for delivering the SiRNA that could be used for treatment.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Methods well known to those skilled in the art can be used to construct expression vectors. One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector.

Another method involves ligation via cohesive ends. Compatible cohesive ends can be generated on the nucleic acid fragment and vector by the action of suitable restriction enzymes. These ends will rapidly anneal through complementary base pairing and remaining nicks can be closed by the action of ligases.

Synthetic linkers containing a variety of restriction endonuclease sites are commercially available from a number of sources including International Biotechnologies Inc, New Haven, Conn., USA.

In one embodiment preferably the composition is a cucurbitacin. Cucurbitacin, C32H46 O8, is a photochemical mostly isolated from cucurbits such as squash. Cucurbitacin Q inhibits the activation of STAT 3 but not JAK2; Cucurbitacin A inhibits JAK2 but not STAT 3 activation; and Cucurbitacin B, E, and I, inhibit the activation of both. Conversion of the C3 carbonyl of the cucurbitacins to a hydroxyl results in loss of anti-JAK2 activity, whereas addition of a hydroxyl group to C11 of the cucurbitacins results in loss of anti-STAT 3 activity. Cucurbitacin Q inhibits selectively the activation of STAT 3 and induces apoptosis without inhibition of JAK2, Src, Akt, Erk, or JNK activation. Furthermore, Cucurbitacin Q induces apoptosis more potently in human and murine tumors that contain constitutively activated STAT3 as compared to those that do not (Blaskovich et al. 2005). By inhibiting STAT phosphorylation the STAT dimer may not be able to move to the nucleus inhibiting the STAT from binding to DNA and inhibiting the regulation of genes via such a pathway.

Preferably the cucurbitacin comprises cucurbitacin I or Cucurbitacin Q. Cucurbitacin I was identified (JSI-124) from the National Cancer Institute Diversity Set to suppress the levels of phosphotyrosine STAT 3 in v-Src-transformed NIH 3T3 cells and human cancer cells potently and rapidly, within 1 to 2 hours. The suppression of phosphotyrosine STAT 3 levels resulted in the inhibition of STAT 3 DNA binding and STAT 3-mediated but not serum response element-mediated gene transcription. Cucurbitacin I also decreased the levels of tyrosine-phosphorylated Janus kinase (JAK) but not those of Src. Cucurbitacin I was highly selective for JAK/STAT 3 and did not inhibit other oncogenic and tumor survival pathways such as those mediated by Akt, extracellular signal-regulated kinase 1/2, or c-Jun NH(2)-terminal kinase. Cucurbitacin I potently inhibited growth in models which express high levels of constitutively activated STAT 3, but it did not affect the growth of oncogenic tumors that are STAT 3 independent (Blaskovich et al. 2003). Compared to inhibition in cancer cells, there may be a higher sensitivity of Cucurbitacin I in fibroproliferative cells. Inhibition of both Jak2 and Stat3 by Cucurbitacin I may exert a more potent inhibitory effect on cell proliferation and migration, compared to inhibition of STAT 3 expression alone.

In one embodiment preferably the composition comprises a STAT 3 decoy oligonucleotide. The STAT 3 decoy comprises a 15-mer double-stranded oligonucleotide, which corresponds closely to the Stat3 response element within the c-fos promoter. The STAT 3 decoy binds specifically to activated Stat3 and blocks binding of Stat3 to a STAT 3 binding element. The STAT 3 decoy oligonucleotide sequence is:

SEQ ID 5: 5′CATTTCCCGTAAATC3′ 3′GTAAAGGGCATTTAC5′

In one embodiment preferably the composition is a phosphotyrosyl peptide and more preferably the phosphotyrosyl peptide comprises XY*L or AY*L. Small molecule phosphotyrosyl peptides inhibitors of Stat3 disrupt the ability of the Stat3 SH2 domain-binding peptide, PY*LKTK (where Y* represents phosphotyrosine). The presence of PY*LKTK, but not PYLKTK or PFLKTK, in nuclear extracts results in significant reduction in the levels of DNA binding activities of STAT 3, to a lesser extent of STAT 1, and with no effect on that of STAT 5. Analyses of alanine scanning mutagenesis and deletion derivatives of PY*LKTK reveal that the Leu residue at the Y+1 position and a substituent at the Y−1 position (but not necessarily Pro) are essential for the disruption of active STAT 3, thereby mapping the minimum active sequence to the tripeptide, XY*L. Studies involving bead-coupled PY*LKTK peptide demonstrate that this phosphopeptide directly complexes with STAT 3 monomers in vitro, suggesting that PY*LKTK disrupts STAT 3:STAT 3 dimers. As evidence for the functional importance of peptide-directed inhibition of STAT 3, PY*LKTK-mts (mts, membrane translocating sequence) selectively inhibits constitutive and ligand-induced STAT 3 activation in vivo. Furthermore, PY*LKTK-mts suppresses transformation by the Src oncoprotein, which has been shown previously to require constitutive STAT 3 activation. XY*L is a minimal peptide that inhibits STAT 3 signaling.

A STAT 3 SH2 domain-binding phosphopeptide, PY*LKTK, and its tripeptide derivatives, PY*L and AY*L (where Y* represents phosphotyrosine) inhibit STAT 3 biochemical activity and biological function. Based on variations of PY*L (or AY*L) with substitution of the Y-1 residue by benzyl, pyridyl, or pyrazinyl new derivatives were 5-fold more potent in disrupting STAT 3 activity in vitro than the PY*L or AY*L tripeptides (Turkson et al. 2001).

In one embodiment preferably the composition is STA-21 a small low molecular weight composition. STA-21 inhibits Stat3 DNA binding activity, Stat3 dimerization, and Stat3-dependent luciferase activity. Moreover, STA-21 reduces the survival of breast carcinoma cells with constitutive Stat3 signaling but has minimal effect on the cells in which constitutive Stat3 signaling is absent (Song et al. 2005).

In one embodiment preferably the composition comprises a composition that modulates STAT 3 as described above and a pharmaceutically acceptable carrier. Preferably, the composition is a unit dosage containing a daily dose or unit, daily sub-dose or an appropriate fraction thereof, of the active ingredient.

Generally, in humans, oral or topical administration of the compositions is the preferred route, being the most convenient. In circumstances where the recipient suffers from a swallowing disorder or from impairment of drug absorption after oral administration, the drug may be administered parenterally, e.g. sublingually or buccally. The compositions of the invention will normally be administered topically or by any parenteral route, in the form of a pharmaceutical composition comprising the active ingredient, optionally in the form of a non-toxic organic, or inorganic, acid, or base, addition salt, in a pharmaceutically acceptable dosage form. Depending upon the fibroproliferative disease and patient to be treated, as well as the route of administration, the compositions may be administered at varying doses.

In human therapy, the compositions can be administered alone but will generally be administered in admixture with a suitable pharmaceutical excipient diluent or carrier selected with regard to the intended route of administration and standard pharmaceutical practice.

In one embodiment preferably the composition is suitable for topical application to a patient. For application topically to the skin, the compositions can be formulated as a suitable ointment containing the active composition suspended or dissolved in, for example, a mixture with one or more of the following: mineral oil, liquid petrolatum, white petrolatum, propylene glycol, polyoxyethylene polyoxypropylene composition, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. The composition may be applied topically in the form of a lotion, solution, cream, ointment or dusting powder.

In one embodiment preferably the pharmaceutical composition further comprises a silicone gel. This may be in the form of medical grade polysiloxane composition sheeting. It may be reusable. It may be a substantially water free composition in the form of a liquid gel. The silicone gel may be in the form of an admixture with the composition or the silicone gel may be placed on the patient after the application of the composition.

In another aspect of the invention a kit of parts comprising a composition that modulates a STAT and a silicone gel. Preferable the composition modulates STAT 3 as described above.

Compositions suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

The compositions of the invention can also be administered parenterally, for example, intravenously, intra-arterially, intraperitoneally, intrathecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral compositions under sterile conditions is readily accomplished by standard pharmaceutical techniques well-known to those skilled in the art.

Compositions suitable for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

In one embodiment preferably the pharmaceutical composition further comprises a corticosteroid. Parenteral administration with Corticosteroid such as triamcinolone acetonide or another corticosteroid medicine may typically be repeated at intervals of four to six weeks. The Corticosteroid may be in the form of an admixture with the composition or the corticosteroid may be administered perenterally to the patient after the application of the composition.

In another aspect of the invention a kit of parts comprising a composition that modulates a STAT and a corticosteroid. Preferably the composition modulates STAT 3 as described above.

The compositions may also be transdermally administered, for example, by the use of a skin patch. In one embodiment preferably the composition may be administered transdermally. Transdermal administration may be via membranes, patches or sheets placed on the patients skin. The membranes may be designed for slow release application of the composition, which may include corticosteroids or other admixtures. The membranes may also be designed to have the advantage of a substantially water free composition. The preparation of suitable membrane compositions under sterile conditions is readily accomplished by standard transdermal techniques well-known to those skilled in the art.

In one embodiment the composition comprises a formula suitable for aerosol delivery to a patient. The compositions of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active composition, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a composition of the invention and a suitable powder base such as lactose or starch.

Aerosol or dry powder compositions are preferably arranged so that each metered dose or “puff” contains at least 1 mg of a composition of the invention for delivery to the patient. It will be appreciated that the overall daily dose with an aerosol will vary from patient to patient, and may be administered in a single dose or, more usually, in divided doses throughout the day. Aerosol administration may be particularly suitable for patients with pulmonary fibrosis.

For example, the compositions of the invention can be administered orally, buccally or sublingually in the form of tablets, capsules, ovules, elixirs, solutions or suspensions, which may contain flavouring or colouring agents, for immediate-, delayed- or controlled-release applications. The preparation of suitable oral compositions under sterile conditions is readily accomplished by standard pharmaceutical techniques well-known to those skilled in the art. The compositions of invention may also be administered via intracavernosal injection. Alternatively, the compositions of the invention can be administered in the form of a suppository or pessary.

Compositions may also be administered by the ocular route, particularly for treating fibroproliferative diseases of the eye. For ophthalmic use, the compositions of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

A further aspect of the invention comprises a method for identifying a composition expected to be useful for treating a fibroproliferative disease, the method comprising the steps of: treating a fibroproliferative cell with a test composition; and assessing the effect of the test composition on STAT.

The method preferably comprises the step of selecting a composition that modulates STAT. More preferably the composition modulates one or more of; STAT activity, phosphorylation of STAT; the level of mRNA or protein expression of STAT; or sub-cellular localisation of the STAT. The composition that modulates the activity of STAT may be selected. For example, a composition that decreases the activity of STAT may be selected or may be a starting point for further investigations or compound design. The composition selected should more preferably reduce the activity of STAT 3 mediated signalling. Modulation of STAT may be assessed using the methods described in the examples below or by those commonly used in the art such as the methods in the papers referred to on pages 4 and 5 above.

In one embodiment preferably STAT is STAT 3. In a further embodiment preferably the fibroproliferative cell is derived from human keloid tissue.

The method of the invention may further comprise the steps of providing, synthesising, purifying and/or formulating a composition selected using computer modelling, as known by those in the art; and of assessing whether the composition modulates the activity of STAT. The composition may be formulated for pharmaceutical use, for example for use in in vivo trials in animals or humans.

In one embodiment preferably assessing the effect of the test composition further comprises assessing the amount or activity of a polypeptide regulated by STAT. The ability of the composition to modulate the activity or level of a protein such as collagen or genes that can be up regulated by STAT 3 such as cyclin D1, Myc, Mcl-1, SOCS3, Bc12, and Bcl-xL may be assessed. For example, activity or level of Myc may be assessed using known assays. Such assessment may also be carried out in a microtitre plate format or other format suitable for high throughput screening. The assessment may be carried out using enzyme assay techniques well known to those skilled in the art.

As noted above, the selected or designed composition may be synthesised (if not already synthesised) or purified and tested for its effect on STAT. The composition may be tested in an in vitro screen for its effect on a Collagen expression that may include Collagen I and or Collagen III either at the mRNA level and or at the polypeptide level or on a cell or tissue in which collagen is present. The cell or tissue may contain endogenous collagen and/or may contain exogenous collagen (including collagen expressed as a result of manipulation of endogenous nucleic acid encoding collagen). The composition may be tested in an ex vivo or in vivo screen, which may use a transgenic animal or tissue. Suitable tests will be apparent to those skilled in the art and examples include more biologically relevant or pathway assessments eg measurement of cell proliferation, production of fibrotic polypeptides; reporter gene activity.

Compositions may also be subjected to other tests, for example toxicology or metabolism tests, as is well known to those skilled in the art.

In one embodiment preferably the effect of the test composition further comprises assessing the amount of cell proliferation, and or the amount of cell migration. Inhibitors of STAT 3 were able to suppress the collagen production, cell proliferation and migration of keloid fibroblasts, to a comparable level to normal fibroblasts.

A further aspect of the invention provides for a method of aiding assessment of a patients risk of developing a fibroproliferative disease, or assessing the severity of a fibroproliferative disease, comprising the step of measuring the level of, STAT expression or STAT activity in a sample.

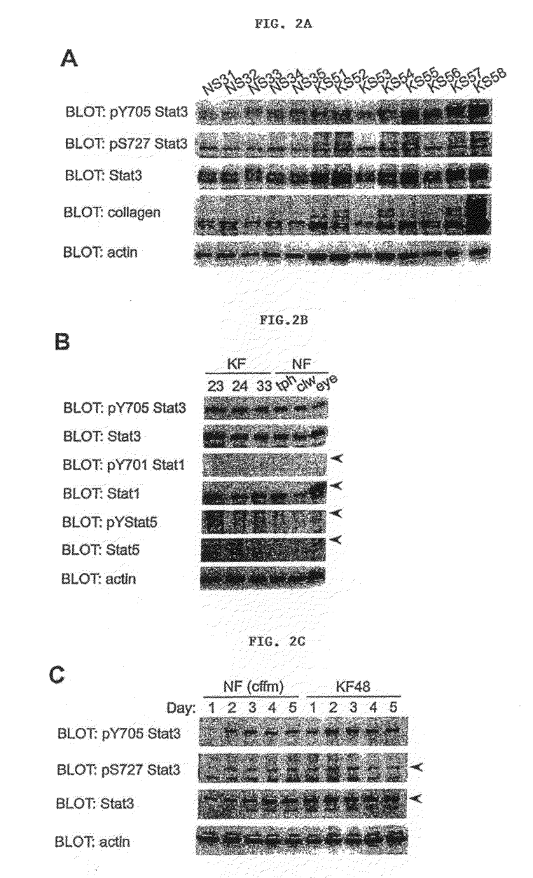

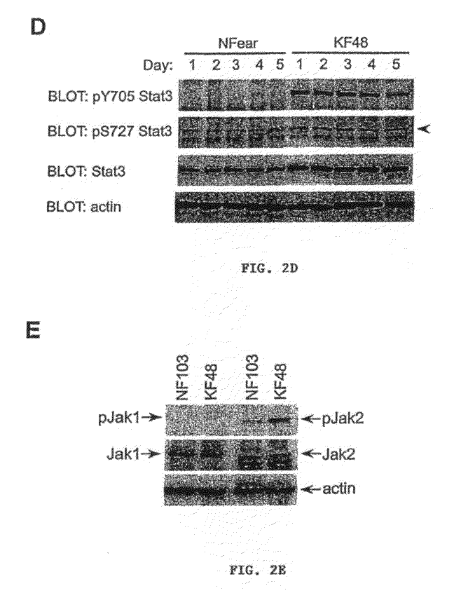

A preferred embodiment provides for determining whether the level indicates that a patient has a low, a medium or a high risk of developing a fibroproliferatinve disease for example after surgery or injury. FIG. 2 A is an example of low (NS31, NS32, NS33, NS34, NS35), medium (KS53, KS56) or high (KS51, KS52, KS54, KS55, KS57, KS58) risk patients that can be determined from the level of STAT expression and activity.

In one embodiment the sample has been obtained from the patient a short time after surgery, but before it can otherwise be determined if the scar will form into a keloid.

In one embodiment the sample has been obtained from a scar. Particularly if there is uncertainty as to whether the scar is hypertrophic or keloid as defined above. Preferably STAT is STAT 3.

The invention will now be described with reference to the following none limiting figures and examples.

All references herein mentioned are hereby incorporated by reference.

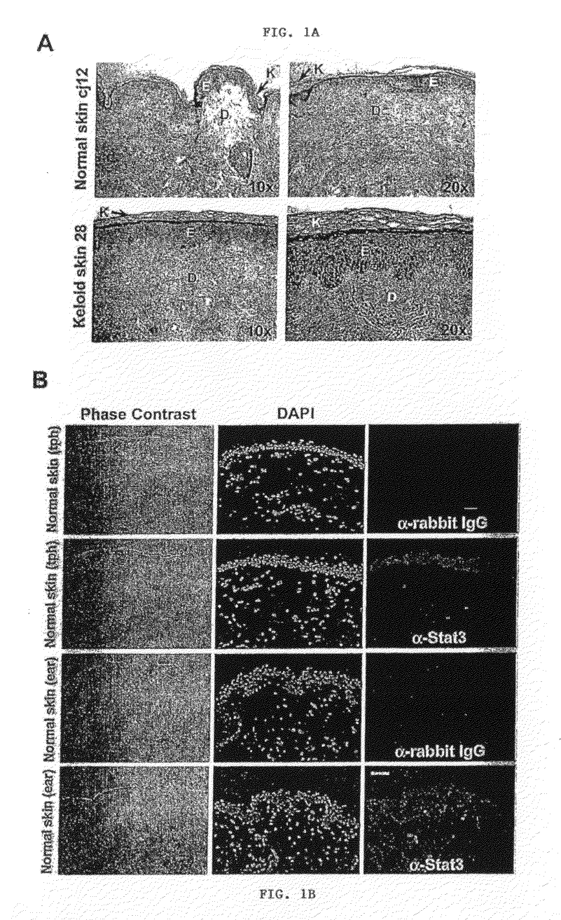

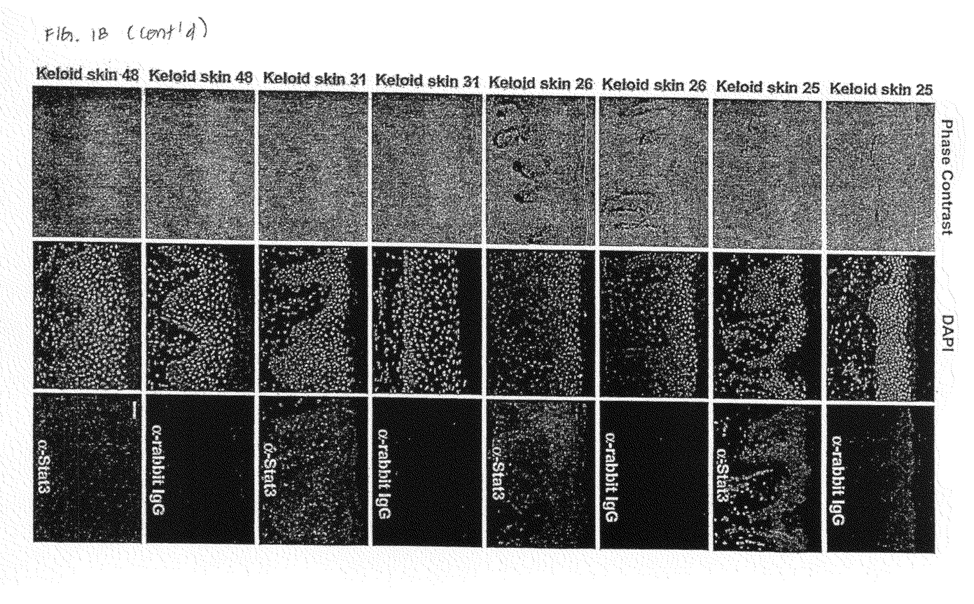

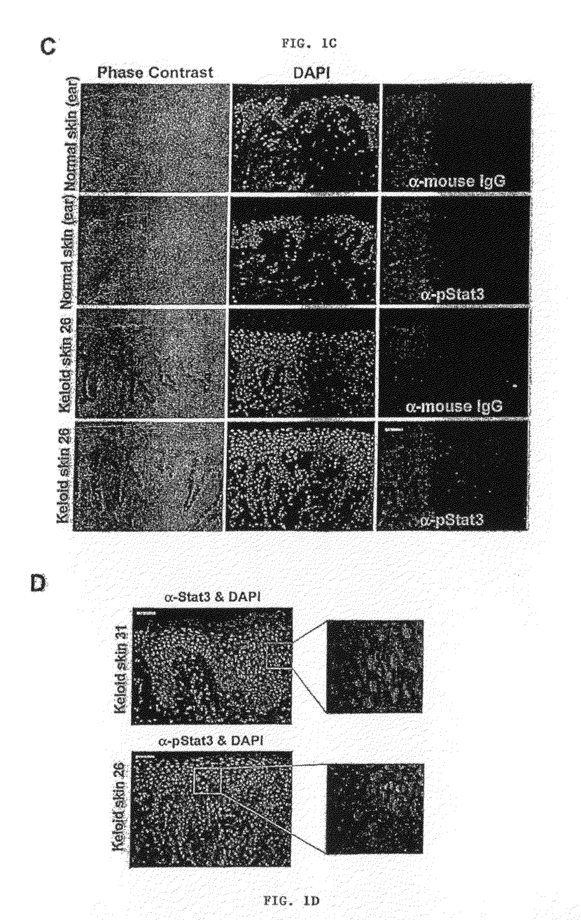

FIG. 1

Stat3 expression and activation are elevated in keloid skin tissues. (A) H&E stain of paraffin tissue sections of normal skin (NS1) and keloid scar (KS28). D, E and K indicate dermis, epidermis, and keratin, respectively. (B) Cryosections of normal skin and keloid scar tissue were stained with either anti-rabbit IgG (α-rabbit IgG) or polyclonal anti-Stat3 (C-20) (a-Stat3) and counterstained with DAPI. (C) Cryosections were probed with anti-mouse IgG (a-mouse IgG) or monoclonal anti-pStat3 (a-pStat3) and counterstained with DAPI. (D) Counterstain between anti-Stat3 (C-20) (a-Stat3) or anti-pStat3 (a-pStat3) with DAPI shows nuclear colocalization of Stat3 and pStat3. Pictures were taken together with phase contrast and all scale bars represent 50 μM.

FIG. 2

Enhanced Stat3 phosphorylation and expression in keloid tissue lysates and keloid fibroblasts. (A) Tissue lysates from normal skin (NS) and keloid samples (KS) were subjected to Western blot analysis with anti-pY705 Stat3 antibody. The blot was stripped and reprobed with anti-pS727 Stat3, and subsequently with anti-Stat3, anti-collagen I, II, III, and anti-actin. (B) Western blot analysis of total cell lysates from normal fibroblasts (NF) and keloid fibroblasts (KF) with antibodies as indicated. The graph on the right illustrates the pY705Stat3 level normalized to Stat3 expression. (C) Normal fibroblast, NF7, and keloid fibroblast, KF48, were cultured in serum-free media for 2 days before stimulating with 10% FBS. Total cell lysates were harvested at day 1 to day 5 and Western blot analysis was performed as mentioned in (A). (D) NF4 and KF48 fibroblasts were cultured in serum-free DMEM for 2 days before harvesting daily over 5 days to investigate the serum-independent phosphorylation of Stat3. Total cell lysates were subjected to Western blot analysis with antibodies as indicated. Arrowheads indicate the position of the protein bands.

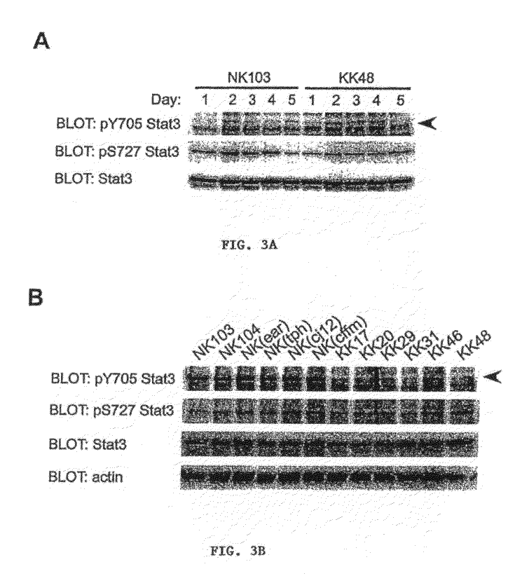

FIG. 3

Stat3-phosphorylation by Jak2 but not Jak1. Total cell lysates from normal fibroblast, NF103, and keloid fibroblast, KF48, were resolved in SDS-PAGE and Western blot analysis was performed using phosphoJak1 (pJak1) or phosphoJak2 (pJak2) antibodies. The blot was stripped and reprobed with Jak1 and Jak2 antibodies respectively, and subsequently with anti-actin for normalization. Arrows indicate the position of the protein bands.

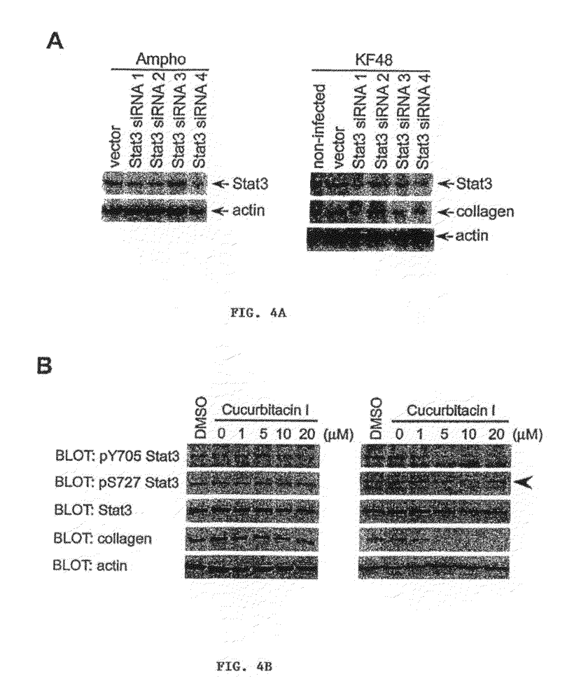

FIG. 4

Increased Tyr705 Stat3 phosphorylation in proliferating, but not in differentiating, keloid keratinocytes. (A) Normal keratinocyte, NK103, and keloid keratinocyte, KK48, were cultured in growth media until nearly confluent, and cells were harvested daily over 5 days. Arrowhead indicates the position of the pY705Stat3 protein. The bands below it in KK48 samples and the strong bands in NK103 are non-specific. (B) Six normal keratinocytes (NK) and six keloid keratinocytes (KK) were cultured in growth media before subjecting the keratinocytes to stratify and undergo terminal differentiation. Western blot analyses were performed as described in FIG. 2.

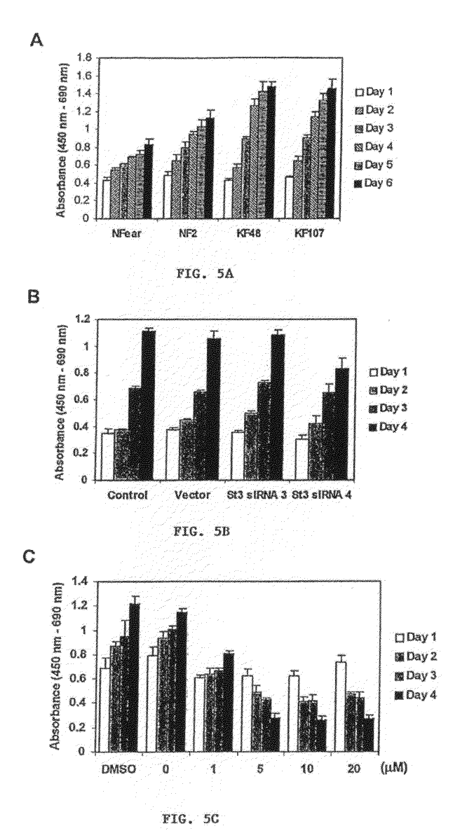

FIG. 5

Inhibition of Stat3 decreases collagen production. (A) Four targets of Stat3 siRNA were cloned into pSuper.retro.puro vector and transfected into amphotropic 293T-based retroviral packaging cell line, including vector alone. Retrovirus harvested 48 h after transfection was used to infect KF48. Total cell lysates from both amphotropic packaging cell line (left panel) and KF48 (right panel) were harvested and analyzed for Stat3, actin and collagen expressions in Western blot analyses. (B) KF48 were left untreated, or treated with DMSO or various doses of Cucurbitacin I (5 μM, 10 μM, 20 μM, or 30 μM) for 30 min, followed by further incubation in 10% FBS/DMEM for 48 h before harvesting. Total cell lysates resolved in SDS-PAGE were examined for the phosphorylation and expression of Stat3, expression of actin, and production of collagen, in Western blot analyses. Arrows/arrowheads indicate the position of the protein bands.

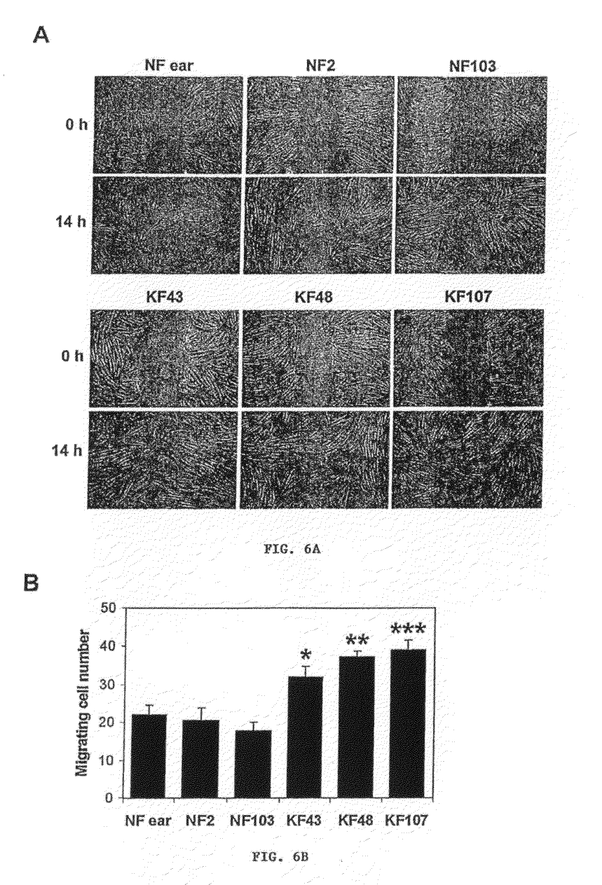

FIG. 6

Inhibition of Stat3 decreases cell proliferation. (A) Increase cell proliferation in keloid fibroblasts compared to normal fibroblasts. Normal fibroblasts, NF2 and NF4, and keloid fibroblasts, KF48 and KF107, were seeded at 3,000 cells/well in 96-well plates in quadruplicates in normal growth media, and cell proliferation was examined daily up to 6 days using XTT cell proliferation kit according to manufacturer's instruction. (B) KF48 fibroblasts were seeded in 96-well plates, infected with retroviral Stat3 siRNAs the next day for 18 h, before changing to 10% FBS/DMEM. Cell proliferation was examined daily from the day of infection. (C) KF48 fibroblasts were seeded in 96-well plates and either left untreated, or treated with DMSO or various doses of Cucurbitacin I, and cell proliferation was examined daily over 4 days.

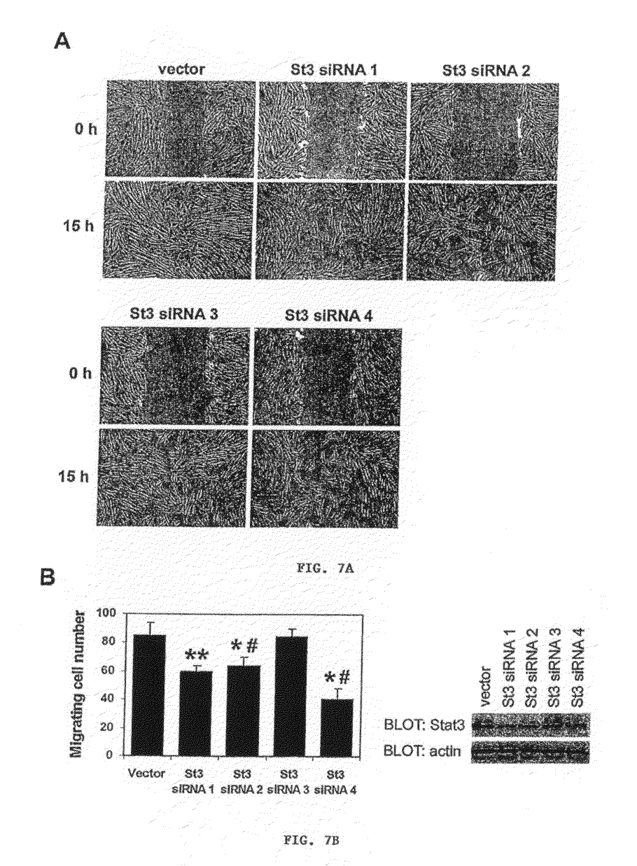

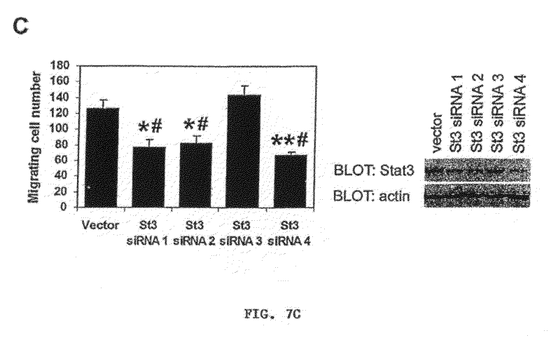

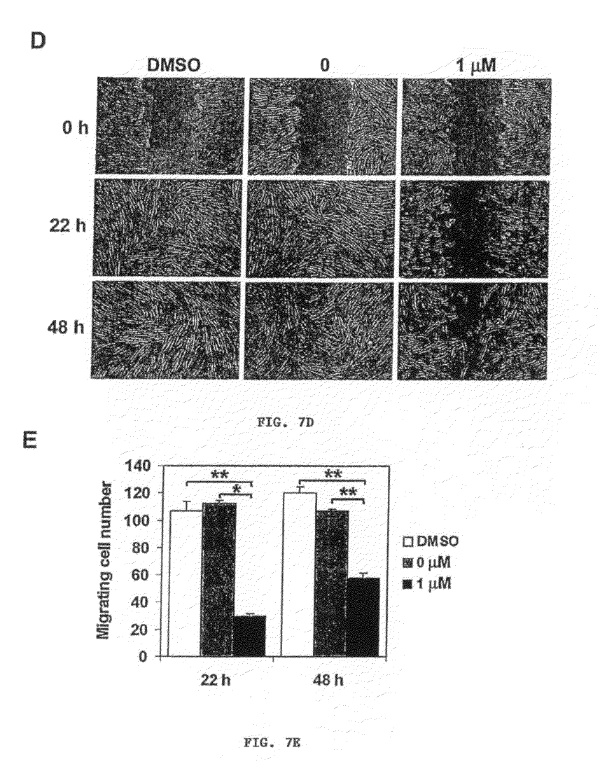

FIG. 7

Increase cell migration in keloid fibroblasts compared to normal fibroblasts. (A) Three normal fibroblasts, NF2, NF4 and NF103, and three keloid fibroblasts, KF43, KF48 and KF107, were grown in 60 mm dishes until confluent, and treated with 10 μg/ml mitomycin C for 2 h, before a scratch wound was introduced using a yellow pipette tip. Cells were incubated for a further 14 h in normal growth media. Pictures were taken at 0 h and 14 h after wounding. (B) Pictures from three non-overlapping fields from (A) were taken and cells migrated into the wound site were quantitated and data are presented as mean±SD. * P<0.05, ** P<0.005 and *** P<0.001, refer to three NFs vs. KF43, KF48 and KF107, respectively, by Student's t-test. (C) Cell migration was also examined using Transwell assay, performed in triplicates. KF48 cells were seeded in serum-free DMEM at the top of the insert with 10% FBS/DMEM as chemoattractant in the bottom chamber. Cells from three non-overlapping fields from each replicate that migrated from top to bottom of the insert after 48 h were counted and the mean of the triplicates±SD was presented in the graph. * P<0.001 and ** P<0.005 indicate three NFs vs. KF43, and KF48 and KF107, respectively, by Student's t-test.

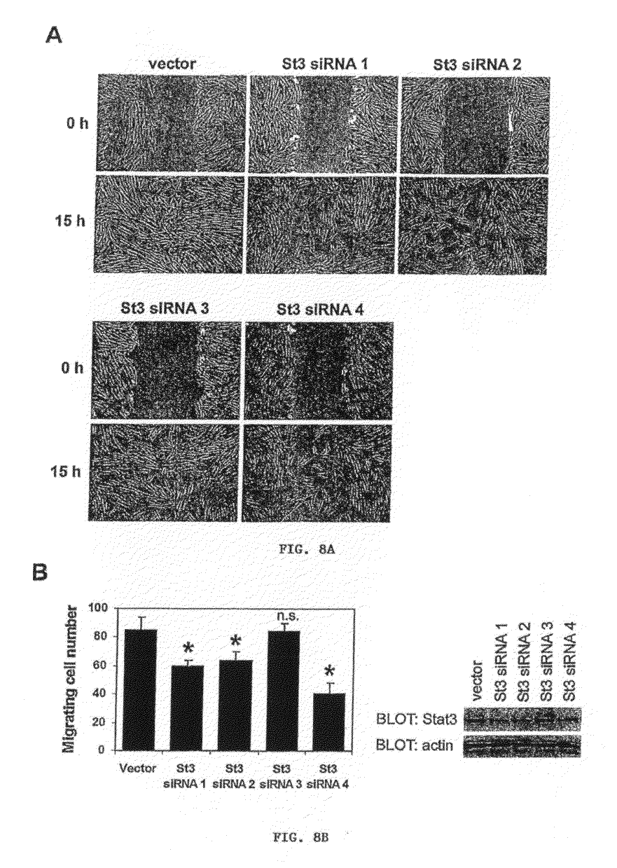

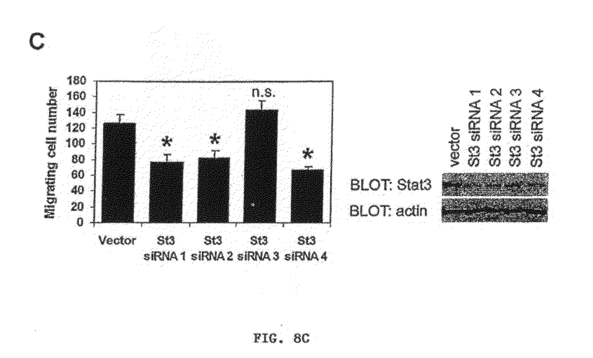

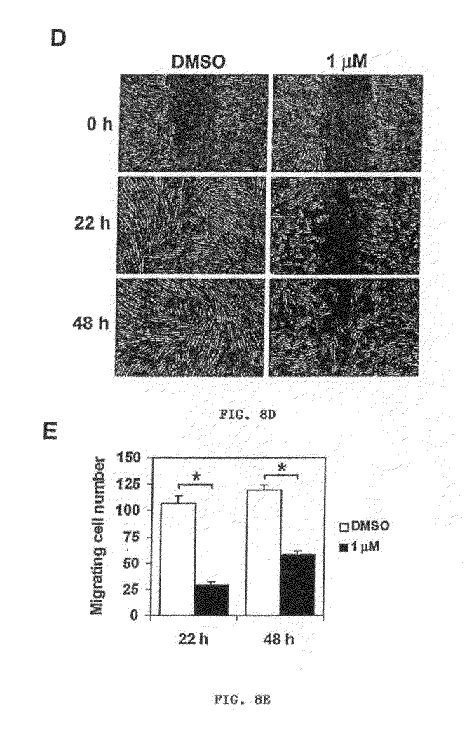

FIG. 8

Decrease cell migration due to inhibition of Stat3. (A) KF48 cells were infected with retrovirus expressing Stat3 siRNAs, and scratch-wound assay was performed as previously described in FIG. 7A. Pictures were taken at 0 h and 15 h after wounding. (B) Data represent mean±SD of cells migrated into the wounding site from three non-overlapping fields from (A). * P<0.005 refers to Stat3 siRNA 1, 2 and 4 vs. vector, by Student's t-test, and n.s. indicates not significant. Inhibition of Stat3 by Stat3 siRNAs in Western blot is shown at the right panel. (C) KF48 cells were infected with retrovirus harboring Stat3 siRNAs and similar cell migration assay using Transwell inserts was performed as mentioned in FIG. 7C. *P<0.001 in comparison to vector by Student's t-test, and n.s. indicates not significant. Inhibition of Stat3 by Stat3 siRNAs is shown in Western blot at the right panel. (D) KF48 cells were pretreated with 10 μg/ml mitomycin C for 2 h, followed by either DMSO or 1 μM Cucurbitacin I for 30 min, before cells were wounded using yellow pipette tips. Cells were further incubated in 10% FBS/DMEM normal growth media, and pictures were taken at 0 h, 22 h and 48 h after wounding. (E) Data represent mean±SD of cells migrated into wounding sites from three non-overlapping fields from (D). * P<0.001 by Student's t-test.

TABLE 1
Profiles of Normal and Keloid Skin Tissue/Fibroblasts/Keratinocytes study.

| Tissue/Fibroblasts/ | | | | | |
|---|---|---|---|---|---|
| Keratinocytes | M/F | Race | Age of “Donor | Origin | Age of Scar |
| NORMAL | | | | | |
| NS/NK (cj12) | F | Chinese | 12 years old | Groin | |
| NS/NF/NK (tph) | F | Chinese | 54 years old | Breast | |
| NS/NF/NK (ear) | M | Chinese | 3 years old | Earlobe | |
| NF (clw) | F | Chinese | 35 years old | Breast | |
| NF (eye) | F | Chinese | 18 years old | Eyelid | |
| NF/NK (cffm) | M | Caucasian | 30 years old | Groin | |
| NF2 | M | Indian | 38 years old | Abdominal | |
| NS31 | F | Malay | 28 years old | Breast | |
| NS32 | M | Chinese | 20 years old | Forearm | |
| NS33 | M | Chinese | 29 years old | Thigh | |
| NS34 | F | Indian | 30 years old | Abdominal | |
| NS35 | F | Indian | 33 years old | Breast | |
| NF/NK103 | M | Chinese | 8 months old | Hand | |
| NK104 | M | Malay | 4 years old | Groin | |
| KELOID | | | | | |
| KK17 | F | Malay | 21 years old | Earlobe | 0.5 yaers |
| KK20 | M | Chinese | 38 years old | Shoulder (L) | 0.5 years |
| KF23 | M | Indian | 37 years old | Chest | 1 year |
| KF24 | F | Chinese | 23 years old | Earlobe | 1.5 years |
| KS25 | M | Indian | 38 years old | Face | 1.5 years |
| KS26 | F | Chinese | 16 years old | Earlobe | 2 years |
| KS28 | F | Chinese | 29 years old | Abdominal | 1.5 years |
| KK29 | M | Indian | 32 years old | Arm (L) | 1 year |
| KS/KK31 | F | Chinese | 17 years old | Earlobe | 0.5 years |
| KF33 | F | Chinese | 19 years old | Earlobe | 1.5 years |
| KF43 | M | Chinese | 34 years old | Elbow | 1 year |
| KK46 | M | Chinese | 36 years old | Forearm | 1 year |
| KS/KF/KK48 | F | Indian | 23 years old | Earlobe | 1.5 years |
| KS51 | F | Indian | 17 years old | Earlobe | 1 year |
| KS52 | F | Indian | 18 years old | Earlobe | 1.5 years |
| KS53 | F | Chinese | 19 years old | Earlobe | 1 year |
| KS54 | M | Malay | 27 years old | Chest | 1 year |
| KS55 | F | Chinese | 22 years old | Earlobe (R) | 1 year |
| KS56 | F | Chinese | 22 years old | Earlobe (L) | 1 year |
| KS57 | M | Indian | 28 years old | Forearm | 1 year |
| KS58 | M | Malay | 22 years old | Earlobe | 1 year |
| KF107 | M | Malay | 11 years old | Face | 1 year |

Normal skin (NS),
keloid skin (KS),
normal fibroblasts (NF),
keloid fibroblasts (KF),
normal keratinocytes (NK), and
keloid keratinocytes (KK) that were presented in this study.

### EXAMPLE 1

We observed enhanced expression and phosphorylation of STAT 3 in epidermal and dermal cells in keloid tissues, and in keloid fibroblasts. Inhibition of STAT 3 expression and phosphorylation by siRNA or Cucurbitacin I resulted in concurrent loss of collagen production, impaired cell proliferation and delayed cell migration in keloid fibroblasts. We show for the first time, a role of STAT 3 in keloid pathogenesis, and novel regulation of collagen production. Inhibitors of STAT 3 may be useful therapeutics for the prospective treatment of keloid scars and presumably other fibroproliferative diseases. In this example, we addressed whether STAT 3 is involved in keloid scar pathogenesis. Moreover, STAT 3 is a key molecule activated by various cytokine and growth factors, ligands which are known to be secreted by various cell components in response to tissue injury during wound healing. Our results showed an enhancement of STAT 3 expression and/or phosphorylation in keloid tissues, keloid-derived fibroblasts and keratinocytes in proliferative condition. We detected concomitant increased activation of tyrosine kinases Jak2, but not Jak1, in keloid fibroblasts compared to normal dermal fibroblasts. In addition, we observed increased collagen production by keloid fibroblasts, cell proliferation and migration in keloid fibroblasts, which were suppressed by STAT 3 inhibition through STAT 3 siRNA and an inhibitor of Jak2/STAT 3, Cucurbitacin I. These data revealed an important role of STAT 3 in the pathogenesis of keloid scars, which could be ameliorated by inhibition of STAT 3, and may be useful as a therapeutic target for the treatment of keloid fibrosis.

Keloid scars do not occur spontaneously, but are the result of excessive wound healing after cutaneous injuries, exhibiting abnormalities in cell migration, proliferation, inflammation, synthesis and secretion of collagen and other ECM components, and remodeling of the wound matrix. We investigated the role of STAT 3 in keloid pathogenesis in both normal skin and keloid tissue sections, as well as in fibroblast and keratinocyte cell cultures. Our data showed that in keloid tissue, STAT 3 is phosphorylated in the stratum basale, but less so in the more superficial differentiated layers of the epidermis (FIG. 1C). This is substantiated by enhanced Tyr705 STAT 3 phosphorylation in keloid keratinocytes maintained in the proliferative rather than in differentiating condition (FIG. 4). In the dermis, we showed that both STAT 3 expression and phosphorylation are enhanced in keloid tissue sections, tissue lysates and fibroblasts (FIGS. 1B, 1C, and 2). Increased collagen production, fibroblast proliferation and migration, have previously been reported in keloid scars. Hence, we further investigated whether the increased STAT 3 expression and phosphorylation we observed has a role in any of these processes in keloid pathogenesis, by means of STAT 3 siRNA or by an inhibitor of Jak2/Stat3 activation, Cucurbitacin I. Indeed, we found that inhibition of Stat3 phosphorylation and expression by either method decreased the keloid fibroblast collagen production, proliferation and migration, to a level comparable to that of normal fibroblasts (FIG. 5-8). STAT 3 siRNA 4 was the most effective among the siRNAs tested, and Cucurbitacin I concentration as low as 1 μM was effective in decreasing the rates of keloid fibroblast proliferation and migration to that of normal fibroblasts. Although higher doses of Cucurbitacin I was efficient in inhibiting Tyr705 and Ser727 STAT 3 phosphorylations and collagen production (FIG. 5B), we also observed cell morphology changes and the beginning of some cell death at higher doses (FIGS. 6C and 8D). As has been previously reported, treatment of A549 human lung carcinoma cells with 10 M Cucurbitacin I for 24 h induced 33% tumor cell death and 9.8% apoptosis, and also 55.4% inhibition of A549 tumor growth in nude mice (Sun et al. 2005). However, compared to these cancer cells, we observed a higher sensitivity of Cucurbitacin I in our primary cells, even at 1 μM exposure for 30 min. Cucurbitacin I was shown to be a more potent inhibitor of pJak2 than pSTAT 3, and we observed increased pJak2 but not pJak1 activation in keloid fibroblasts compared to normal fibroblasts (FIG. 3). Inhibition of both Jak2 and STAT 3 by Cucurbitacin I seemed to exert a more potent inhibitory effect on cell proliferation and migration, compared to inhibition of STAT 3 expression alone by siRNA.

The process of cutaneous wound healing can be arbitrarily divided into three phases—inflammation, tissue formation (reepithelialization, formation of granulation tissue, neovascularization) and tissue remodeling, events which are governed by a well-orchestrated temporal and spatial secretion of various chemokines, cytokines and growth factors by different cell components. STAT 3 is a key molecule activated in response to most of these ligands. Here, we observed enhanced expression and phosphorylation of STAT 3 in keloid tissues, fibroblasts and keratinocytes, suggesting a lack of a stop signal or its downregulation, and/or an incessant stimulation by cytokines or growth factors. Indeed, some cytokines that activate STAT 3 have been reported to be enhanced in keloid tissue such as IL-6 (Xue et al. 2000). On the other hand, whether there is any mutation in STAT 3 in keloid tissues that predisposes to its overexpression remains to be determined. Since Stat3 is involved in all the different phases of wound healing, we speculate that normal regulation of Stat3 is required throughout the process, and dysregulation of Stat3 signaling will tip the balance towards impaired wound healing and fibrosis. Activation of Stat3 by proinflammatory cytokines also suggests a role of Stat3 in the progression of scarring at the inflammation phase, since fetal tissues that lack inflammation undergo perfect scarless wound healing, and scarring ensued in the presence of an acute inflammatory infiltrate (Yang et al. 2003).

Fibroblasts are responsible for the synthesis, deposition, and remodeling of the ECM. We report here that overexpression and phosphorylation of Stat3 plays a novel role in the excessive collagen deposition by fibroblasts, leading to fibrotic tissue such as keloids. Inhibition of STAT 3 expression by siRNA or Cucurbitacin I showed a corresponding decline in collagen expression (FIG. 5), suggesting that collagen production may be regulated by STAT 3 at the transcriptional level. Preliminary scanning of the promoter region of both collagen type I al, COL1A1, and collagen type III al, COL3A1, revealed some putative STAT 3 binding sites. It will be interesting to investigate whether Stat3 has a direct effect on the transcription of procollagen type I and III, the two collagens that are found highly synthesized in keloids.

STAT 3 was shown to possess oncogenic potential, as overexpression of a constitutively-dimerized/active STAT 3 mutant (STAT 3C) caused cellular transformation and tumor formation. Recently, overexpression of STAT 3C in A549 carcinoma cells followed by microarray analyses revealed that STAT 3 regulated genes are common to both wound healing and cancer, including cell invasion/migration, angiogenesis, and remodeling of ECM, based on the concept that tumors are “wounds that do not heal”. This supports our observation that hyperactive STAT 3 plays a role in fibroproliferative disease or wound healing gone awry, as seen in keloids. However, there is still a distinct difference between wound healing and skin cancer, as cancer cells metastasize and scars do not. It is still unclear how STAT 3 plays a differential role between wound healing and skin cancer. Keratinocyte-specific STAT 3-deficient mice exhibited impaired wound healing, suggesting that either expression and/or activation of Stat3 in keratinocytes plays a critical role in that process. On the other hand, introduction of the active STAT 3C into keratinocytes spontaneously led to psoriasis in mice by 2 weeks (Sono et al. 2005). However, the psoriatic lesions developed by tape-stripping wounding require both the activated Stat3 in keratinocytes and the presence of activated T lymphocytes, demonstrating that active STAT 3 in keratinocytes alone is insufficient to impair wound healing. We have shown that STAT 3 is hyperactive in keloid fibroblasts and proliferating keratinocytes, but it remains to be determined if T cells are also required for keloid scarring. As for skin cancer, STAT 3 was completely resistant to the development of skin tumor in the keratinocyte-specific Stat3-deficient mice subjected to a two-step model of chemically-induced skin carcinogenesis, and STAT 3 inhibitor applied topically into transgenic mice expressing v-Ha-ras in keratinocytes inhibited the papilloma formation (Chan et al. 2004 and Pedranzini et al. 2004). The involvement of T cell activation in tumor progression was not addressed in these reports, and further investigation using T cell-deficient nude mice will be required to answer this question.

Recently, unphosphorylated STAT 3 has been proposed in oncogenesis and transcriptional regulation. We observed strong expression of STAT 3 in both keratinoctyes and fibroblasts in normal and keloid tissues, and superimposition with the nuclear stain, DAPI, showed strong nuclear localization of STAT 3. Yet, with the PSTAT 3 staining, we could not determine just how much of the STAT 3 was phosphorylated. While it has been very well established that pTyr705 STAT 3 translocates to the nucleus, binds to target DNA sequences and regulates gene transcription, the role of unphosphorylated Stat3 is less clear. We consider that unphosphorylated STAT 3, cytoplasmic or nuclear, may play a role in keloid pathogenesis, but most likely in a different manner than that of the pTyr705 STAT 3 subpopulation. However, the mechanism remains ambiguous and warrants further investigation.

In summary, we have shown for the first time, a novel role of STAT 3 in the pathogenesis of keloid scars. Inhibitors of STAT 3 expression or phosphorylation were able to suppress keloid fibroblast collagen production, cell proliferation and migration to a level comparable to normal fibroblasts. Besides siRNA and Cucurbitacin I, other STAT 3 inhibitors are considered to be useful, including other pharmacological agents such as Cucurbitacin Q which is an analogue of Cucurbitacin I and a selective STAT 3 inhibitor (Sun et al. 2005), STA-21 (Song et al. 2005), STAT 3 decoy oligonucleotide (Leong et al. 2003), or phosphotyrosyl peptides (Turkson et al. 2001), all of which exhibited either anti-tumor activity, increased apoptosis, inhibited proliferation, or suppressed cell transformation of cancer-derived or v-Src-transformed cell lines. These potential cancer therapeutic agents that target the inhibition of STAT 3 or any other compositions that inhibit STAT 3 are also considered to be useful for the prospective clinical treatment of fibroproliferative disease including keloid scars.

### Characterisation of Fibroproliferative Tissue

Normal and keloid tissue. Ethical approvals from National University of Singapore Institutional Review Board and National Healthcare Group Domain Specific Review Boards, and informed consent from patients, were obtained before surgical excision of skin and keloid tissue. All patients had received no prior treatment for keloid lesions. A full history was taken and physical examination was performed in addition to color-slide photodocumentation of all keloid lesions. Normal skin and keloid scar tissue mentioned in this paper are listed in Table 1.

Cell culture. Normal and keloid-derived fibroblasts and keratinocytes mentioned in this work are listed in Table 1. Isolated fibroblasts were grown in DMEM containing 4,500 mg/ml glucose, supplemented with 10% FBS (GIBCO BRL Life Technologies, NY, USA), 2 mM L-glutamine, 100 U/ml penicillin and 100 ng/ml streptomycin (Sigma, Mo., USA). Isolated keratinocytes were cultured in either Keratinocyte Growth Media (KGM; Clonetics, NY, USA) or EpiLife medium (Cascade Biologics Inc., OR, USA). To allow keratinocytes to stratify and reach terminal differentiation, cells in serum-free KGM were maintained till 100% confluent in monolayer, followed by DMEM supplemented with 10% FCS for 4 days, before subjecting them to air-liquid interface (Lim et al. 2001).

Enhanced Stat3 expression and phosphorylation in keloid scar tissue versus normal skin tissue in vivo. In normal skin, a thin layer of epidermis underlies a very thin layer of keratin. In contrast, the keratin and epidermal layers of keloid tissue are thicker, as shown by the H&E staining of paraffin sections (FIG. 1A). The dermis is also considerably thicker, with compacted and irregular connective tissue compared to normal skin which appears more uniformly stacked and regular.

Haematoxylin and eosin staining. Tissue sections were fixed with 4% paraformaldehyde in PBS, embedded in paraffin and sectioned. Tissue sections were stained with Haematoxylin for 5-8 min, washed with running water, quick destained with acid ethanol containing 1% hydrochloric acid and 70% enthanol, washed with running water, and counterstained with eosin-ethanol for 3-5 min. Tissue sections were then rehydrated by gradual immersion in 70%, 80%, 95% and 100% ethanol, cleared with xylene, and finally mounted in entellan. Pictures were taken with Leica DM4000 B microscope.

Immunofluorescence. Cryo sections of the tissue samples were fixed with 4% paraformaldehyde in PBS for 15-30 min at room temperature, washed thrice with 0.1% Triton X-100 in PBS for 10 min each, and blocked with buffer containing 5% normal goat serum, 2% fetal bovine serum, 2% bovine serum albumin, 1 mM CaCk, 1 mM MgC2 in PBS. Tissue sections were washed as above and incubated for 1 h at room temperature, with primary antibody diluted 1:200 in FDB to 1 μg/ml for monoclonal p-Stat3 (B7), polyclonal Stat3 (C-20), anti-mouse IgG, or anti-rabbit IgG antibodies. Tissue sections were washed again as described and incubated for 1 h at room temperature, with either FluoroLink Cy3-labelled goat anti-rabbit IgG (PA43004; Amersham Biosciences, Buckinghamshire, UK) or anti-mouse IgG-Cy3 (AP124C; Chemicon International, Temecula, Calif., USA) diluted at 1:400 in FDB. Sections were washed again and mounted with Vectashield mounting medium containing DAPI (H-1200; Vector Laboratories, Inc., Burlingame, Calif., USA). Pictures were taken with Leica DM4000 B microscope.

### Assessment of STAT Modulation

Assessment of STAT expression. To examine the expression of Stat3, cryosections from normal skin and skin overlying keloid scar were subjected to immunofluorescence using a polyclonal Stat3 (C-20) antibody, performed in parallel with anti-rabbit IgG as controls. As illustrated in FIG. 1B, Stat3 could be detected in both normal skin and keloid tissue, whereas weak fluorescence was observed with anti-rabbit IgG antibody. Stat3 expression was easily detected in the epidermal layer in both skin types, which is predominantly populated with keratinocytes. In the dermal layer, the connective tissue is interspersed with cellular components comprised primarily of fibroblasts, which are involved in the deposition of collagen fibers and other ECM components. A small population of fibroblast cells in the dermal layer of normal skin 3 and 4 also showed Stat3 expression, whereas a higher number of fibroblasts in keloid samples 25, 26, 31 and 48 showed Stat3 expression. The Stat3 immunofluorescence appeared more intense in keloid compared to normal skin, indicating a higher Stat3 expression in the cell population in both epidermis and dermis in keloid samples.

Retroviral Stat3 siRNA. Four different targets designed against STAT 3 were selected and cloned into BglII and XhoI into pSUPER.retro.puro from OligoEngine, and sequenced (STAT 3 Genbank Accession number NM_{—}139276). The four targets are named STAT 3 siRNA 1 (nt. 461-480) SEQ ID 1, STAT 3 siRNA 2 (nt. 1264-1283) SEQ ID 2, STAT 3 siRNA 3 (nt. 364-383) GATTGGGCATATGCGGCCA, and STAT 3 siRNA 4 (nt. 1662-1681) SEQ ID 3. The corresponding primers with a 9-nt short hairpin in the middle (bold) are

SEQ ID 4:
F15′GATCCCCAGTCGAATGTTCTCTATCATTCAAGAGATGATAGAGAA
CATTCGACTTTTTTC3′
and
SEQ ID 5:
5′TCGAGAAAAAAGTCGAATGTTCTCTATCATCTCTTGAATGATAGAG
AACATTCGACTGGG3′,
SEQ ID 6: F2 5′GATCCCC
GGCGTCCAGTTCACTACTATTCAAGAGATAGTAGTGAACTGGACGCCT
TTTTC3′
and
SEQ ID 7:
5′TCGAGAAAAAGGCGTCCAGTTCACTACTATCTCTTGAATAGTAGTG
AACTGGACGCCGGG3′,
siRNA 3
5′GATCCCCGATTGGGCATATGCGGCCATTCAAGAGATGGCCGCA
TATGCCCAATCTTTTTC3′
and
5′TCGAGAAAAAGATTGGGCATATGCGGCCATCTCTTGAATGGCCGCA
TATGCCCAATCGGG3′,
SEQ ID 8: F4
5′GATCCCCGCGTCCATCCTGTGGTACATTCAAGAGATGTACCACAGG
ATGGACGCTTTTTC3′,
and
SEQ ID 9:
5′TCGAGAAAAAGCGTCCATCCTGTGGTACATCTCTTGAATGTACCAC
AGGATGGACGCGGG3′.

These vectors containing Stat3 siRNA targets and empty vector were transfected into 293T-based Phoenix-Ampho packaging cell line for 7-9 h, and the amphotropic retroviruses were harvested 48 h later, pelleted to remove nonadherent cells and cellular debris, and filtered through a 0.45 μM cellulose acetate membrane. The keloid fibroblasts were infected overnight in the presence of 4 μg/ml polybrene, and replaced with fresh 10% FBS/DMEM the following day. Cells were assayed for Western blot 48 h after infection, or for XTT cell proliferation, or for migration assay which was performed by replating the cells into 8 μM Transwell membrane and fixed 24 h later as described above.

Of the four different targets for Stat3 siRNA selected, three displayed an inhibitory effect on Stat3 expression, namely Stat3 siRNA 1, 2 and 4, but not siRNA 3, in 293T-based amphotropic packaging cell line (FIG. 5A, left panel).

Assessment of STAT phosphorylation, activity, and sub-cellular localisation. Activation of Stat3 was also examined using phosphoStat3 (pStat3) monoclonal antibody, performed in parallel with anti-mouse IgG as controls. Weak pStat3 staining was observed in normal skin 4 (FIG. 1C). In contrast, activation of Stat3 was significantly increased in both dermis and epidermis in keloid 26.

Assessing sub-cellular localization. Superimposing the Stat3 stain with nuclear DAPI stain in keloid 31 showed colocalization of the dot-like Stat3 stain with the nucleus (FIG. 1D, upper panels), with amorphous stain indicating cytoplasmic localization. This reinforced the suggestion that more activated Stat3 was present in keloid-derived cells. Similar nuclear localization was also observed in keloid 26, stained with anti-pStat3 antibody and DAPI (FIG. 1D, lower panels). Closer examination of keloid 26 revealed that nuclear staining of Stat3 was detected predominantly in the stratum basale, with less staining in both the stratum spinosum and stratum granulosum layers. This indicates that activation of Stat3 is more enhanced in keloid keratinocytes compared to normal keratinocytes in the stratum basale adjacent to the basement membrane, suggesting a role of activated Stat3 in cell proliferation rather than cell differentiation in keloid keratinocytes.

Increased Stat3 activation in keloidfibroblasts and tissue lysates compared to normal skin fibroblasts and tissue lysates. To further verify the data above, tissue lysates of normal skin from five individuals and keloid scars from eight individuals were investigated for phosphorylation and expression of Stat3. Three samples from normal skin showed low Tyr705 Stat3 phosphorylation, whereas two showed moderate Tyr705 Stat3 phosphorylation (FIG. 2A). In contrast, all keloid tissue lysates, except for one, showed moderate to very high Tyr705 Stat3 phosphorylation. The degree of Tyr705 Stat3 phosphorylation correlated well with the level of Stat3 expression. In addition, six out of eight keloid tissue samples showed an increase in Ser727 Stat3 phosphorylation compared to all five normal skin samples. Collagen production was elevated in the same six of eight keloid tissue samples, whereas the remaining two showed a similar expression to those from the normal skin samples.

The epidermis is populated with 95% keratinocytes and 5% non-keratinocyte cells comprising melanocytes, Langerhans cells, and Merkel cells (33), whereas fibroblasts are the predominant cells in the dermis, along with some endothelial and mast cells. To further investigate the activation of Stat3, primary fibroblasts derived from normal skin and keloid tissues were further examined. Two normal fibroblast (NF) and three keloid fibroblast (KF) strains were examined for Stat3 activation. Tyr705 phosphorylation of Stat3 was elevated from 2.3 to 4.3-fold in keloid fibroblasts compared to normal fibroblasts after normalization with Stat3 expression (FIG. 2B). Tyr phosphorylation of Stat1 and Stat5 were undetectable in any of the samples. Stat1 protein expression was present, whereas Stat5 expression was weak, in all of the samples.

To further examine the kinetics of Tyr705 and Ser727 Stat3 phosphorylation, normal and keloid fibroblasts were cultured for 5 days under normal growth conditions. In normal fibroblast, NF7, Tyr705 Stat3 phosphorylation was hardly detectable at day 1, but was observed at day 2 and sustained until day 5 (FIG. 2C). In contrast, Tyr705 Stat3 phosphorylation was observed in keloid fibroblast, KF48, at day 1, which increased and peaked at day 3, and gradually decreased to day 1 levels at day 5. The Ser727 Stat3 phosphorylation profile was similar to Tyr705 Stat3 phosphorylation in both normal and keloid fibroblasts. In addition, an elevated Stat3 expression was observed in KF48 compared to NF7. This data correlated well with the observations seen in tissue sections and tissue lysates (FIG. 1B-D and FIG. 2A).

Stat3 phosphorylation was also examined in serum-free condition. NF4 and KF48 were cultured to 90% confluence, washed extensively with serum-free DMEM, and further incubated in serum-free DMEM for 2 days. Fresh serum-free DMEM was added, and cells were harvested every 24 h up to day 5. We observed strong constitutive Tyr705 and Ser727 Stat3 phosphorylation in KF48 throughout the 5 days, with very weak phosphorylation in NF4 (FIG. 2D). Although actin expression was similar in all samples, Stat3 expression was slightly elevated in KF48 compared to NF4. Overall, phosphorylations and expression of Stat3 were augmented in keroid fibroblasts and tissue lysates compared to the samples from normal skin. Tyr705 and Ser727 Stat3 phosphorylations in keloid fibroblasts also occur in a constitutive and serum-independent manner.

Jak2 but not Jak1 activation is increased in keloid fibroblasts. The enhanced Tyr705 Stat3 phosphorylation in keloid fibroblasts prompted us to investigate the activation of known Tyr kinases of Stat3, namely Jaks, Src and EGF receptor. Nearly confluent normal and keloid fibroblasts were maintained in serum-free media and harvested, and activation of endogenous Jak1 and Jak2 was examined in Western blot analysis using anti-phosphoJak1 or anti-phosphoJak2 antibody. As shown in FIG. 3, increased pJak2 was detected in KF48 compared to NF103, whereas pJak1 signal was hardly detectable in either normal or keloid fibroblasts, although Jak1 expression was present in both cell types. On the other hand, phosphoSrc (Tyr416) and phosphoEGFR (Tyr845) signals were hardly detectable in both NF and KF (data not shown).

Inhibition of Stat3 was also evaluated by incubating KF48 for 30 min with various doses of Cucurbitacin I, an inhibitor of Jak2/Stat3 activation (32), or with DMSO alone as a control, followed by further incubation in normal growth media for 48 h. As shown in FIG. 5B, a dose-dependent inhibition of pY705 Stat3, pS727 Stat3, and collagen production was observed by Cucurbitacin I, with effective concentration as low as 1 μM. A dose-dependent decrease of Stat3 expression was also observed, although actin expression was rather similar in all samples. Cucurbitacin I was acquired from Galchimia (A Corufia, Spain).

Stat3 activation in keloid keratinocytes. Stat3 activation was also examined in keratinocyte cell lines derived from normal and keloid tissue. Keratinocytes were cultured in growth medium and harvested daily up to day 5. Tyr705 Stat3 phosphorylation was detectable in KK48 (FIG. 4A, middle band indicated by arrowhead), but not in NK103. On the other hand, Ser727 Stat3 phosphorylation was higher in NK103 compared to KK48 over days 1 to 4, with a reversal of phosphorylation level at day 5. Stat3 immunoblot showed equal expression between NK103 and KK48.

Keratinocytes grown in growth media represent keratinocytes of the stratum basale in the proliferative phase. The results above are consistent with the pStat3 staining in the stratum basale in keloid 26 (FIG. 1C). To further investigate whether Stat3 activation occurred in the other stratified layers of epidermis in vivo, keratinocytes were cultured in differentiation condition. Interestingly, while Tyr705 Stat3 phosphorylation was comparatively high in all of the six normal keratinocyte samples, it was significantly weak in all six keloid keratinocyte samples examined (FIG. 4B). In addition, 50% of the normal keratinocytes displayed a slightly enhanced Ser727 Stat3 phosphorylation compared to keloid keratinocytes, while Stat3 and actin expressions were similar in all samples. The decreased Tyr705 Stat3 phosphorylation in differentiating keloid keratinocytes was also consistent with poor pStat3 staining in both stratum spinosum and granulosum in keloid 26 (FIG. 1C).

### Antibodies and Reagents

Antibodies to all 7 STAT family members including a panel of phospholelation specific antibodies to STAT 1, STAT 3, and STAT 6 are commercially available form Acris Antibodies GmbH, (Hiddenhausen Im Himmelreich, Germany). All of these can be used for western blotting and some can be used for immunohistochemical staining. In addition a number of other companies have one or more STAT antibodies available.

Polyclonal anti-phosphoTyr705 Stat3, monoclonal antiphosphoSer727 Stat3, polyclonal anti-phosphoJak1 (Tyr1022/1023) and polyclonal antiphosphoJak2 (Tyr1007/1008) antibodies were purchased from Cell Signaling (Beverly, Mass., USA), whereas monoclonal pStat3 (B7) (sc-8059), polyclonal Stat3 (C-20) (sc-482) antibody, anti-mouse IgG (sc-2025), and anti-rabbit IgG (sc-2027) were acquired from Santa Cruz Biotechnology, Inc. (Santa Cruz, Calif., USA). Monoclonal anti-Stat3 antibody was purchased from BD Transduction Laboratories (Lexington, Ky., USA), and polyclonal anti-actin antibody was purchased from Sigma (Saint Louis, Mo., USA).

Western blot. Cells were either harvested in radioimmune precipitation assay (RIPA) buffer (150 mM NaCl, 50 mM Tris-HCl, pH 7.2, 1% deoxycholic acid, 1% Triton X-100, 0.25 mM EDTA, pH 8.0) containing protease inhibitor cocktail (Roche Diagnostics Corporation, Indianapolis, Ind., USA), or lysis buffer containing 150 mM NaCl, 10 mM Tris-HCl, pH 7.4, 0.5% Triton-X-100, 0.5% NP-40, 1 mM EDTA, 0.2 mM sodium vanadate, supplemented with protease inhibitor tablet. Total cell lysates were collected after centrifugation for 10 min at 4° C., and resolved in 7.5% or 10% polyacrylamide gel electrophoresis, and transferred to PVDF membranes (Bio-Rad Laboratories, Hercules, Calif., USA). The membranes were blotted with anti-phosphoTyr705 Stat3 antibody (1:1000 dilution; Cell Signaling, Beverly, Mass., USA) overnight at 4° C. overnight in PBS containing 1% BSA and 0.1% Tween 20, washed with PBS containing 0.2% Tween 20, and incubated with secondary anti-rabbit IgG antibody (1:2000 dilution; Sigma, Saint Louis, Mo., USA) for 1 h at room temperature, washed with PBS containing 0.2% Tween 20, followed by enhanced chemiluminescence (Amersham Biosciences, Buckinghamshire, UK) or Lumi-Light (Roche Diagnostics Corporation, Indianapolis, USA), and autoradiography. The membranes were stripped with Restore Western Blotting Stripping Buffer (Pierce, Rockford, Ill., USA) according to manufacturer's instructions and reblotted with anti-phosphoSer727 Stat3 (1:1000 dilution; Cell Signaling, Beverly, Mass., USA) and secondary anti-mouse IgG antibody (1:2000 dilution; Sigma, Saint Louis, Mo., USA). The stripping and reblotting procedure was repeated with monoclonal anti-Stat3 (1:1000 dilution; BD Transduction Laboratories), polyclonal anti-actin (1:1000 dilution; Sigma) and monoclonal anti-collagen antibody (1:1000 dilution; MONOSAN).

Fibroproliferative Cells Derived from Human Keloid Tissue

Isolation of fibroblasts and keratinocytes. The procedure for the isolation and culture of fibroblasts and keratinocytes from skin specimens was as described previously (Lim et al. 2002).

Methods of assessing polypeptides regulated by STAT Collagen may be regulated by STAT 3 activation. monoclonal anti-human collagen I, II, III Was purchased from MONOSAN (Uden, Netherlands). Fibronectin and mitomycin C were purchased from Sigma (Saint Louis, Mo., USA). These were detected via western blot as described above.

Collagen production was elevated in six of eight keloid tissue samples (FIG. 2A), whereas the remaining two showed a similar expression to those from the normal skin samples.

Inhibition of Stat3 downregulates collagen production. Secretion of collagen by fibroblasts occurs during the process of tissue remodeling in wound healing, and enhanced collagen production has been implicated in fibrosis. To investigate whether STAT 3 plays any role in keloid collagen production, keloid fibroblasts were infected with retrovirus expressing STAT 3 SiRNAs. Of the four different targets for Stat3 siRNA selected, three displayed an inhibitory effect on Stat3 expression, namely Stat3 siRNA 1, 2 and 4, but not siRNA 3, in 293T-based amphotropic packaging cell line (FIG. 5A, left panel). Inhibition of Stat3 expression by these three Stat3 siRNAs correlated with the inhibition of collagen production in KF48 (FIG. 5A, right panel). Taken together, inhibition of Stat3 expression by Stat3 siRNA, or Stat3 activation by Cucurbitacin I, resulted in equivalent inhibition of collagen production.

### Methods of Assessing Cell Migration

Cell migration in keloidflbroblasts is inhibited by Stat3 siRNA and Cucurbitacin I. To examine the role of Stat3 in cell migration of skin fibroblasts, KF48 cells were infected with retrovirus expressing Stat3 siRNAs and scratch assays were performed as previously described. In FIG. 8A, Stat3 siRNA 1, 2 and 4, all of which inhibit Stat3 expression, were able to impede the rate of cell migration of KF48, whereas vector alone or Stat3 siRNA 3 did not. Three non-overlapping fields were acquired, and graphically illustrated as mean±SD in FIG. 8B. Transmembrane cell migration assays were also performed in triplicates as described earlier, and quantified as mean±SD (FIG. 8C). In agreement with the scratch assay, Stat3 siRNA 1, 2 and 4, were able to hinder the cell migration of KF48, whereas vector and Stat3 siRNA 3 did not. The right panels in FIG. 8B and 8C depict immunoblots of Stat3 and actin to show the efficiencies of inhibition by the Stat3 siRNAs.

The role of Stat3 in cell migration was also examined using Cucurbitacin I in the scratch assay. KF48 cells were first incubated with 10 μg/ml mitomycin C for 2 h, before treating for 30 min with DMSO or 1 μM Cucurbitacin I. The scratch-wound was performed, followed by further incubation in normal growth media. Pictures were taken at 0 h, 22 h and 48 h after wounding. As shown in FIG. 8D, inhibition of Stat3 activation by Cucurbitacin I decelerated the migration of KF48, compared to DMSO control. Quantification of the mean±SD of three non-overlapping fields is depicted in FIG. 8E, which showed a significant reduction in cell migration at 22 h and a delayed migration at 48 h by Cucurbitacin I compared to DMSO control (*P<0.001).

Increased cell migration in keloidfibroblasts. Wound healing is a complex multi-step process. It involves the migration and proliferation of keratinocytes and fibroblasts to resurface areas of skin loss. We further examined the migration potential of normal and keloid fibroblasts using the scratch assay. Cells were first cultured to full confluence, and treated with 10 μg/ml mitomycin C for 2 h before scratch-wounding with a yellow pipette tip. Pictures were taken at 0 h and 14 h later (FIG. 7A). Three non-overlapping fields were captured and quantitated as mean±SD, and results are presented in FIG. 7B. All three keloid fibroblasts, KF43, KF48 and KF107, showed enhanced cell migration rates towards the midline of scratch wound compared to three normal fibroblasts, NF2, NF4 and NF103. Cell migration was also examined by the transmembrane assay using the 8.0 μM pore size polyvinylpropylene-free polycarbonate Transwell membranes, performed in triplicates. Three non-overlapping fields per replicate were captured and quantitated as the mean of the total migrating cells in the triplicates. Once again, the three KF samples showed increased migration compared to the three NF samples (FIG. 7C).

Migration assay. For migration assay using the scratch method, cells were grown to confluence and incubated with 10 μg/ml mitomycin C for 2 h, before a scratch wound was introduced using a yellow pipette tip. Cells were further incubated in normal growth media and pictures were taken at 0 h, 14 h, 15 h, 22 h, or 48 h after wounding. Three non-overlapping fields were captured by Axiovert 135 microscope from Zeiss and cells migrated into the wound site were counted and presented as mean±SD.

Migration assay was also performed using the Transwell 8.0 μM pore size polycarbonate membrane (Corning Inc.). 1×10⁴ cells/well were seeded at the top of the insert in 0.2 ml serum-free DMEM, with 0.5 ml 10% FBS/DMEM in the bottom chamber as chemoattractant. 24 h later, cells were washed once in cold PBS, before fixing with 4% paraformaldehyde in PBS for 15 min at room temperature. Cells were permeabilized with 0.1% Triton X-100 in PBS for 3 min at room temperature, and stained with haematoxylin for 10 min at room temperature, followed by destain with water thrice. Cells from the top of the inserts were removed by cotton swabs, and inserts were left to dry. The inserts were mounted onto slides and three non-overlapping fields of cells migrated to the bottom of the inserts were captured using Leica DM4000 B microscope.

### Methods of Assessing Cell Proliferation

Inhibition of Stat3 decreases cell proliferation. Enhanced fibroblast proliferation is a characteristic of keloid tissue. In agreement with this, keloid fibroblasts grown in normal culture conditions proliferated faster than normal fibroblasts, as observed in KF48 and KF107 compared to NF2 and NF4 (FIG. 6A). To investigate whether Stat3 is involved in the enhanced keloid fibroblast proliferation, KF48 was infected with retrovirus expressing Stat3 siRNAs, and cell proliferation was examined. As shown in FIG. 6B, inhibition of Stat3 by Stat3 siRNA 4 decreased cell proliferation of KF48, compared to a noninfected control, vector control and Stat3 siRNA 3, which does not inhibit Stat3. XTT proliferation assay. Cells seeded at 3,000 cells/well in 96-well plates and grown in normal growth conditions, or infected with retroviral Stat3 siRNAs, were assayed for proliferation using the XrT proliferation kit (Roche) according to the manufacturer's instructions. The readings were taken at 450 nm and referenced against 690 nm, 2 h after the addition of the XrT reagent. Each sample was performed at quadruplicates in two to three independent experiments. Data are presented as mean±SD

Cell proliferation was also investigated by treating KF48 cells with various doses of Cucurbitacin I or with DMSO control, before incubating in normal growth media and examined daily over 4 days. Cucurbitacin I concentration as low as I μM was able to inhibit cell proliferation of KF48 compared to DMSO and untreated control (0 μM), whereas Cucurbitacin I exposure at 5 μM and above seemed to cause cell death (FIG. 6C).

### REFERENCES

Abergel, R. P., Pizzurro, D., Meeker, C. A., Lask, G., Matsuoka, L. Y., Minor, R. R., Chu, M. L., and Uitto, J. 1985. Biochemical composition of the connective tissue in keloids and analysis of collagen metabolism in keloid fibroblast cultures. J Invest Dermatol 84:384-390.
Babu, M., Diegelmann, R., and Oliver, N. 1989. Fibronectin is overproduced by keloid fibroblasts during abnormal wound healing. Mol Cell Biol 9:1642-1650.
Babu, M., Diegelmann, R., and Oliver, N. 1992. Keloid fibroblasts exhibit an altered response to TGF-beta. J Invest Dermatol 99:650-655.
Bhowmick, N. A., Chytil, A., Plieth, D., Gorska, A. E., Dumont, N., Shappell, S., Washington, M. K., Neilson, E. G., and Moses, H. L. 2004. TGF-beta signaling in fibroblasts modulates the oncogenic potential of adjacent epithelia. Science 303:848-851.
Blaskovich, M. A., Sun, J., Cantor, A., Turkson, J., love, R., and Sebti, S. M. 2003. Discovery of JSI-124 (cucurbitacin I), a selective Janus kinase/signal transducer and activator of transcription 3 signaling pathway inhibitor with potent antitumor activity against human and murine cancer cells in mice. Cancer Res 63:1270-1279.
Bromberg, J. F., Wrzeszczynska, M. H., Devgan, G., Zhao, Y., Pestell, R. G., Albanese, C., and Darnell, J. E., Jr. 1999. Stat3 as an oncogene. Cell 98:295-303.
Calderon, M., Lawrence, W. T., and Banes, A. J. 1996. Increased proliferation in keloid fibroblasts wounded in vitro. J Surg. Res 61:343-347.
Chan, K. S., Sano, S., Kiguchi, K, Anders, J., Komazawa, N., Takeda, J., and DiGiovanni, J. 2004. Disruption of Stat3 reveals a critical role in both the initiation and the promotion stages of epithelial carcinogenesis. J Clin Invest 114:720-728.
Cosman, B., Crikelair, G. F., Gaulin, J. C., and Lattes, R. 1961. The surgical treatment of keloids. Plast. Reconstr. Surg. 27:335-358.
Craig, R. D. P., Schofield, J. D., and Jackson, S. S. 1975. Collagen biosynthesis in normal human skin, normal and hypertrophic scar and keloid. Eur J Clin Invest 5:69-74.
Darnell, J. E., Jr. 1997. STATs and gene regulation. Science 277:1630-1635.
Darnell, J. E., Jr., Kerr, I. M., and Stark, G. R. 1994. Jak-STAT pathways and transcriptional activation in response to IFNs and other extracellular signaling proteins. Science 264:1415-1421.
Dauer, D. J., Ferraro, B., Song, L., Yu, B., Mora, L., Buettner, R., Enkemann, S., Jove, R., and Haura, E. B. 2005. Stat3 regulates genes common to both wound healing and cancer. Oncogene 24:3397-3408.
Diegelmann, R. F., Cohen, I. K, and McCoy, B. J. 1979. Growth kinetics and collagen synthesis of normal skin, normal scar and keloid fibroblasts in vitro. J Cell Physiol 98:341-346.
Edmondson, S. R., Thumiger, S. P., Werther, G. A., and Wraight, C. J. 2003. Epidermal homeostasis: the role of the growth hormone and insulin-like growth factor systems. Endocr. Rev 24:737-764.
Gira, A. K., Brown, L. F., Washington, C. V., Cohen, C., and Arbiser, J. L. 2004. Keloids demonstrate high-level epidermal expression of vascular endothelial growth factor. J Am Acad Dermatol 50:850-853.
Haisa, M., Okochi, H., and Grotendorst, G. R. 1994. Elevated levels of PDGF alpha receptors in keloid fibroblasts contribute to an enhanced response to PDGF. J Invest Dermatol 103:560-563.
Lee, T. Y., Chin, G. S., Kim, W. J., Chau, D., Gittes, G. K., and Longaker, M. T. 1999. Expression of transforming growth factor beta 1, 2, and 3 proteins in keloids. Ann. Plast. Surg. 43:179-184.
Leong, P. L., Andrews, G. A., Johnson, D. E., Dyer, K. F., Xi, S., Mai, J. C., Robbins, P. D., Gadiparthi, S., Burke, N. A., Watkins, S. F. et al. 2003. Targeted inhibition of Stat3 with a decoy oligonucleotide abrogates head and neck cancer cell growth. Proc Natl Acad Sci USA 100:4138-4143.
Lim, I. J., Phan, T. T., Bay, B. H., Qi, R, Huynh, H., Tan, W. T., Lee, S. T., and Longaker, M. T. 2002. Fibroblasts cocultured with keloid keratinocytes: normal fibroblasts secrete collagen in a keloidlike manner. Am J Physiol Cell Physiol 283:C212-C222.
Lim, I. J., Phan, T. T., Song, C., Tan, W. T., and Longaker, M. T. 2001. Investigation of the influence of keloid-derived keratinocytes on fibroblast growth and proliferation in vitro. Plast. Reconstr. Surg. 107:797-808.
Mustoe, T. A. 2004. Scars and keloids. BMJ 328:1329-1330. 6. Alster, T. S. and Tanzi, E. L. 2003. Hypertrophic scars and keloids: etiology and management. Am J Clin Dermatol 4:235-243.
Naitoh, M., Hosokawa, N., Kubota, H., Tanaka, T., Shirane, H., Sawada, M., Nishimura, Y., and Nagata, K. 2001. Upregulation of HSP47 and collagen type III in the dermal fibrotic disease, keloid. Biochem Biophys Res Commun 280:1316-1322.
Nencioni A, Sandy P, Dillon C, Kissler S, Blume-Jensen P, Van Parijs L. (2004) RNA interference for the identification of disease-associated genes. Curr Opin Mol Ther. Vol 6(2):136-40.
Pedranzini, L., Leitch, A., and Bromberg, J. 2004. Stat3 is required for the development of skin cancer. J Clin Invest 114:619-622.
Ragoowansi, R., Cornes, P. G., Glees, J. P., Powell, B. W., and Moss, A. L. 2001. Earlobe keloids: treatment by a protocol of surgical excision and immediate postoperative adjuvant radiotherapy. Br. J Plast. Surg. 54:504-508.
Saed, G. M., Ladin, D., Olson, J., Han, X., Hou, Z., and Fivenson, D. 1998. Analysis of p53 gene mutations in keloids using polymerase chain reaction-based single-strand conformational polymorphism and DNA sequencing. Arch. Dermatol 134:963-967.
Sano, S., Chan, K. S., Carbajal, S., Clifford, J., Peavey, M., Kiguchi, K., Itami, S., Nickoloff, B. J., and DiGiovanni, J. 2005. Stat3 links activated keratinocytes and immunocytes required for development of psoriasis in a novel transgenic mouse model. Nat Med 11:4349.
Sano, S., Itami, S., Takeda, K., Tarutani, M., Yamaguchi, Y., Miura, H., Yoshikawa, K., Akira, S., and Takeda, J. 1999. Keratinocyte-specific ablation of Stat3 exhibits impaired skin remodeling, but does not affect skin morphogenesis. EMBO J 18:4657-4668.
Singer, A. J. and Clark, R. A. 1999. Cutaneous wound healing. N Engl J Med 341:738-746.
Song, H., Wang, R., Wang, S., and Lin, J. 2005. A low-molecular-weight composition discovered through virtual database screening inhibits Stat3 function in breast cancer cells. Proc Natl Acad Sci USA 102:47004705.
Sun, J., Blaskovich, M. A., Jove, W, Livingston, S. K., Coppola, D., and Sebti, S. M. 2005. Cucurbitacin Q: a selective STAT3 activation inhibitor with potent antitumor activity. Oncogene 24:3236-3245.
Takeda, K. and Akira, S. 2000. STAT family of transcription factors in cytokinemediated biological responses. Cytokine Growth Factor Rev 11: 199-207.
Takeda, K., Noguchi, K, Shi, W., Tanaka, T., Matsumoto, M., Yoshida, N., Kishimoto, T., and Akira, S. 1997. Targeted disruption of the mouse Stat3 gene leads to early embryonic lethality. Proc Natl. Acad Sci USA 94:3801-3804.
Tuan, T. L. and Nichter, L. S. 1998. The molecular basis of keloid and hypertrophic scar formation. Mol Med Today 4:19-24.
Turkson, J., Ryan, D., Kim, J. S., Zhang, Y., Chen, Z., Haura, E., Laudano, A., Sebti, S., Hamilton A. D., and Jove, R. 2001. Phosphotyrosyl peptides block Stat3-mediated DNA binding activity, gene regulation, and cell transformation. J Biol Chem 276:45443-45455.
Uitto, J., Perejda, A. J., Abergel, R. P., Chu, M. L., and Ramirez, F. 1985. Altered steady-state ratio of type I/III procollagen mRNAs correlates with selectively increased type I procollagen biosynthesis in cultured keloid fibroblasts. Proc Natl Acad Sci USA 82:5935-5939.
Wen, Z., Zhong, Z., and Darnell, J. E., Jr. 1995. Maximal activation of transcription by Stat1 and Stat3 requires both tyrosine and serine phosphorylation. Cell 82:241-250.
Werner, S. and Grose, R. 2003. Regulation of wound healing by growth factors and cytokines. Physiol Rev 83:835-870.
Wu, Y., Zhang, Q., Ann, D. K., Akhondzadeh, A., Duong, H. S., Messadi, D. V., and Le, A. D. 2004. Increased vascular endothelial growth factor may account for elevated level of plasminogen activator inhibitor-1 via activating ERK1/2 in keloid fibroblasts. Am J Physiol Cell Physiol 286:C905-C912.
Xue, H., McCauley, R. L., and Zhang, W. 2000. Elevated interleukin-6 expression in keloid fibroblasts. J Surg. Res 89:74-77.
Yang, J., Chatterjee-Kishore, M., Staugaitis, S. M., Nguyen, H., Schlessinger, K., Levy, D. E., and Stark, G. R. 2005. Novel roles of unphosphorylated STAT3 in oncogenesis and transcriptional regulation. Cancer Res 65:939-947.
Yoshimoto, H., Ishihara, H., Ohtsuru A., Akino, K, Murakami, R., Kuroda, H., Namba, H., Ito, M., Fujii, T., and Yamashita, S. 1999. Overexpression of insulin-like growth factor-1 (IGF-I) receptor and the invasiveness of cultured keloid fibroblasts. Am J Pathol. 154:883-889.
Younai, S., Nichter, L. S., Wellisz, T., Reinisch, J., Nimni, M. E., and Tuan, T. L. 1994. Modulation of collagen synthesis by transforming growth factor-beta in keloid and hypertrophic scar fibroblasts. Ann. Plast. Surg. 33:148-151.

1. Use of a composition that modulates a STAT3 in the manufacture of a medicament for treating or preventing keloid scarring.

2. The use of claim 1 wherein the composition modulates one or more of; activity; phosphorylation; level of expression; or sub-cellular localisation of the STAT3.

3. The use of claim 1 wherein the composition comprises an SiRNA of STAT 3.

4. The use of claim 3 wherein the SiRNA of STAT 3 comprises SEQ ID 1, or SEQ ID 2 or SEQ ID 3.

5. The use of claim 4 wherein the SiRNA of STAT comprises SEQ ID 4 and or SEQ ID 5, or SEQ ID 6 and or SEQ ID 7, or SEQ ID 8 and or SEQ ID 9.

6. The use of claim 5 wherein the SiRNA of STAT is selected from the group of SEQ ID4 and or SEQ ID 5, or SEQ ID 6 and or SEQ ID 7, or SEQ ID 8 and or SEQ ID 9.

7. The use of claim 1 wherein the composition comprises a cucurbitacin.

8. The use of claim 7 wherein the composition comprises cucurbitacin I or cucurbitacin Q.

9. The use of claim 1 wherein the composition comprises a STAT 3 decoy oligonucleotide.

10. The use of claim 1 wherein the composition comprises a phosphotyrosyl peptide.

11. The use of claim 10 wherein the phosphotyrosyl peptide comprises XY*L or AY*L.

12. The use of claim 1 wherein the composition comprises a pharmaceutical composition and a pharmaceutically acceptable carrier.

13. The use of claim 12 wherein the composition is suitable for topical application to a patient.

14. The use of claim 12 wherein the composition is suitable for transdermal administration to a patient.

15. The use of claim 12 wherein the composition is suitable for parenteral administration to a patient.

16. The use of claim 12 wherein the composition is suitable for aerosol administration to a patient.

17. The use of claim 12 wherein the composition comprises a silicone gel or wherein the medicament is for administering to a patient who is also administered a silicone gel.

18. The use of claim 12 wherein the composition comprises a corticosteroid or wherein the medicament is for administering to a patient who is also administered a corticosteroid.

19. A method for identifying a composition expected to be useful for treating keloid scarring, the method comprising the steps of: treating a cell derived from human keloid tissue with a test composition; and assessing the effect of the test composition on STATs.

20. The method of claim 19 wherein the composition modulates STAT3.

21. The method of claim 19 wherein the composition modulates one or more of; activity; phosphorylation; level of expression; or sub-cellular localisation of the STAT3.

22. The method of claim 19 wherein assessing the effect of the test composition comprises assessing the amount or activity of polypeptide regulated by STAT3.

23. The method of claim 19 wherein assessing the effect of the test composition comprises assessing the amount of cell proliferation, and or the amount of cell migration.

24. A kit comprising a composition that modulates a STAT3 and a silicone gel, wherein the composition is suitable for treating or preventing keloid scarring.

25. A kit comprising a composition that modulates a STAT3 and a corticosteroid, wherein the composition is suitable for treating or preventing keloid scarring.

26. The kit of claim 24 wherein the composition modulates one or more of; activity; phosphorylation; level of expression; or sub-cellular localisation of the STAT.

27. The kit of claim 24 wherein the STAT is STAT 3.

28. The kit of claim 24 wherein the composition comprises an SiRNA of STAT 3.

29. The kit of claim 28 wherein the SiRNA of STAT 3 comprises SEQ ID 1, or SEQ ID 2 or SEQ ID 3.

30. The kit of claim 29 wherein the SiRNA of STAT 3 comprises SEQ ID 4 and or SEQ ID 5, or SEQ ID 6 and or SEQ ID 7, or SEQ ID 8 and or SEQ ID 9.

31. The kit of claim 30 wherein the SiRNA of STAT 3 is selected from the group of SEQ ID 4 and or SEQ ID 5, or SEQ ID 6 and or SEQ ID 7, or SEQ ID 8 and or SEQ ID 9.

32. The kit of claim 24 wherein the composition comprises a cucurbitacin.

33. The kit of claim 32 wherein the composition comprises cucurbitacin I or cucurbitacin Q.

34. The kit of claim 24 wherein the composition comprises a STAT 3 decoy oligonucleotide.

35. The kit of claim 24 wherein the composition comprises a phosphotyrosyl peptide.

36. The kit of claim 35 wherein the phosphotyrosyl peptide comprises XY*L or AY*L.

37. A method of aiding assessment of a patient's risk of developing keloid scarring, or assessing the severity of keloid scarring, comprising the step of measuring the level of, STAT3 expression or STAT3 activity in a sample.

38. The method of claim 37 comprising determining whether the level indicates that a patient has a low, a medium or a high risk of developing a fibroproliferatinve disease for example after surgery or injury.

39. The method of claim 38 wherein the sample has been obtained from the patient a short time after surgery.

40. The method of claim 37 wherein the sample comes from a scar.
